(19) United States
(12) Patent Application Publication
(10) Pub. No.: US 2026/0363673 A1
(43) Pub. Date: Nov. 19, 2026

(54) METHOD OF PROVIDING INFORMATION ABOUT CARDIAC ULTRASOUND IMAGE, AND ULTRASOUND IMAGING APPARATUS THEREFOR

(71) Applicant: Samsung Medison Co., Ltd., Hongcheon-gun (KR)

(72) Inventors: Taewoo Kwon, Seoul (KR); Subin Park, Seoul (KR)

(73) Assignee: SAMSUNG MEDISON CO., LTD., Hongcheon-gun (KR)

(21) Appl. No.: 19/650,913

(22) Filed: Apr. 17, 2026

(30) Foreign Application Priority Data
May 14, 2025 (KR) 10-2025-0062832
Sep. 4, 2025 (KR) 10-2025-0126106

(51) Int. Cl. A61B 8/00; A61B 8/08

(52) U.S. Cl. CPC A61B 8/54; A61B 8/0883; A61B 8/463; A61B 8/468

(57) ABSTRACT

Provided are an ultrasound imaging apparatus for providing information about a heart from an ultrasound image, and a method of controlling the ultrasound imaging apparatus. The ultrasound imaging apparatus via an ultrasound transceiver module, an ultrasound signal toward a heart of an object, obtain ultrasound images of a cycle of the heart based on an ultrasound echo signal received from the object via the ultrasound transceiver module, identify regions of a plurality of chambers in the obtained ultrasound images, determine a position of an apex of a first chamber among the plurality of chambers and a position of a valve of the first chamber, based on a relative position between a second chamber and the apex and the valve of the first chamber, calculate information about the heart based on the position of the apex and the position of the valve, and display the calculated information.

### CROSS-REFERENCE TO RELATED APPLICATION

This application is based on and claims priority under 35 U.S.C. § 119 to Korean Patent Application No. 10-2025-0062832, filed on May 14, 2025, in the Ministry of Intellectual Property, Republic of Korea, and Korean Patent Application No. 10-2025-0126106, filed on Sep. 4, 2025, in the Ministry of Intellectual Property, Republic of Korea, the disclosures of which are incorporated by reference herein in their entirety.

### BACKGROUND

### 1. Field

The disclosure relates to an ultrasound imaging apparatus for detecting information about a heart from a cardiac ultrasound image and providing the detected information, a method of controlling the ultrasound imaging apparatus, and a computer-readable recording medium having stored therein a computer program for executing the method of controlling the ultrasound imaging apparatus.

### 2. Description of the Related Art

Recently, in the medical field, various medical imaging apparatuses have become widely used for imaging and obtaining information about living tissues of a human body for early diagnosis or surgery of various diseases. Representative examples of such medical imaging apparatuses may include ultrasound imaging apparatuses, computed tomography (CT) apparatuses, and magnetic resonance imaging (MRI) apparatuses.

An ultrasound imaging apparatus emits an ultrasound signal generated by a transducer of a probe toward an object, and receives information about the signal reflected from the object, so as to non-invasively obtain at least one image of an internal region of the object (e.g., soft tissue or blood flow). An ultrasound imaging apparatus may be used for medical purposes such as observation of an interior of an object, detection of foreign substances, or assessment of injury. Such ultrasound imaging apparatuses offer advantages of higher stability, real-time video display, and safety without radiation exposure compared to imaging apparatuses using X-rays, and thus are widely used alongside other imaging apparatuses.

### SUMMARY

Additional aspects will be set forth in part in the description which follows and, in part, will be apparent from the description, or may be learned by practice of the presented embodiments of the disclosure.

An aspect of the disclosure may provide an ultrasound imaging apparatus. The ultrasound imaging apparatus may include a memory storing instructions, and at least one processor including processing circuitry. The instructions may, when executed by the at least one processor individually or collectively, cause the ultrasound imaging apparatus to emit, via the ultrasound transceiver module, an ultrasound signal toward a heart of an object, obtain ultrasound images of a cycle of the heart based on an ultrasound echo signal received from the object via the ultrasound transceiver module. The ultrasound imaging apparatus may identify regions of a plurality of chambers in the obtained ultrasound images. The ultrasound imaging apparatus may determine a position of an apex of a first chamber among the plurality of chambers and a position of a valve of the first chamber, based on a relative position between a second chamber and the apex and the valve of the first chamber. The ultrasound imaging apparatus may calculate information about the heart based on the position of the apex and the position of the valve. The ultrasound imaging apparatus may display the calculated information.

An aspect of the disclosure may provide a method of providing information about a heart from a cardiac ultrasound image. The method may include emitting an ultrasound signal toward a heart of an object and obtaining ultrasound images of a cycle of the heart based on an ultrasound echo signal received from the object. The method may include identifying regions of a plurality of chambers in the obtained ultrasound images. The method may include determining a position of an apex of a first chamber among the plurality of chambers and a position of a valve of the first chamber, based on a relative position between a second chamber and the apex and the valve of the first chamber. The method may include calculating information about the heart based on the position of the apex and the position of the valve. The method may include displaying the calculated information.

An aspect of the disclosure may provide a computer-readable recording medium having recorded thereon a program for causing a computer to execute the method of providing information about a heart from a cardiac ultrasound image.

### BRIEF DESCRIPTION OF THE DRAWINGS

The disclosure may be readily understood from a combination of the following detailed description and the accompanying drawings, wherein reference numbers refer to structural elements.

FIG. 1A and FIG. 1B are block diagrams illustrating configurations of an ultrasound imaging system according to an embodiment.

FIGS. 2A, 2B, 2C, and 2D are diagrams each illustrating an ultrasound imaging system according to an embodiment.

FIG. 3 illustrates a method, performed by an ultrasound imaging apparatus, of providing information about a heart, according to an embodiment.

FIG. 4 is a flowchart of a method, performed by an ultrasound imaging apparatus, of calculating information about a heart from cardiac ultrasound images, according to an embodiment.

FIG. 5 illustrates a flowchart of a method, performed by an ultrasound imaging apparatus, of calculating information about a heart from cardiac ultrasound images, according to an embodiment.

FIG. 6 illustrates a method, performed by an ultrasound imaging apparatus, of determining the positions of an apex and a valve of a chamber, according to an embodiment.

FIG. 7 illustrates a method, performed by an ultrasound imaging apparatus, of determining an apex and a valve of a chamber, according to an embodiment.

FIG. 8 illustrates a method, performed by an ultrasound imaging apparatus, of adjusting the positions of an apex and a valve based on a user input, according to an embodiment.

FIG. 9 illustrates a method, performed by an ultrasound imaging apparatus, of calculating a longitudinal strain of a chamber, according to an embodiment.

FIG. 10 is a flowchart of a method, performed by an ultrasound imaging apparatus, of identifying a contour of a valve, according to an embodiment.

FIG. 11 illustrates a method, performed by an ultrasound imaging apparatus, of determining a cycle of a heart by considering the contour of a valve, according to an embodiment.

FIG. 12 is a flowchart of a method, performed by an ultrasound imaging apparatus, of providing information about a cycle of a heart, according to an embodiment.

FIG. 13 is a diagram illustrating a method, performed by an ultrasound imaging apparatus, of displaying a plurality of cycles of a heart, according to an embodiment.

FIG. 14 illustrates a method, performed by an ultrasound imaging apparatus, of displaying information about chambers, according to an embodiment.

FIG. 15 illustrates a user interface for cardiac information, according to an embodiment.

FIG. 16 illustrates a method, performed by an ultrasound imaging apparatus, of providing information about a heart according to user settings, according to an embodiment.

### DETAILED DESCRIPTION

To clarify the scope of the claims of the disclosure and to enable those of skill in the art to which embodiments belong to practice the embodiments, the principles of the embodiments will be described and disclosed. The embodiments may be implemented in various forms.

Like reference numerals denote like elements throughout the specification. The specification does not describe all elements of embodiments, and general content in the art to which the disclosure pertains or identical content between the embodiments will be omitted. A “module” or “unit” used herein may be implemented with software, hardware, firmware, or a combination thereof, and depending on embodiments, a plurality of “modules” or “units” may be implemented as one element, or one “module” or “unit” may include a plurality of elements.

A singular form of a noun corresponding to an item may include one or more of the things, unless the relevant context clearly indicates otherwise.

As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases.

As used herein, the term “and/or” includes any one or a combination of a plurality of related recited elements.

As used herein, such terms as “1st” and “2nd,” or “first” and “second” may be used to simply distinguish a corresponding component from another, and does not limit the components in any other respect (e.g., importance or order).

In addition, as used herein, such terms as “front surface”, “rear surface”, “upper surface”, “lower surface”, “side surface”, “left”, “right”, “upper”, or “lower” are defined with respect to the drawings, and the shape and position of each component are not limited by these terms.

As used herein, such terms as “comprises,” “includes,” or “has” specify the presence of stated features, numbers, stages, operations, components, parts, or a combination thereof, but do not preclude the presence or addition of one or more other features, numbers, stages, operations, components, parts, or a combination thereof.

When an element is referred to as being “connected to,” “coupled to,” “supported by,” or “in contact with” another element, it means that the element is directly connected to, coupled to, supported by, or in contact with the other element, or that the element is indirectly connected to, coupled to, supported by, or in contact with the other element via a third element.

When an element is referred to as being “on” another element, it means that the element is in contact with the other element, or that still another element is present between the element and the other element.

Hereinafter, an ultrasound apparatus according to various embodiments will be described in detail with reference to the accompanying drawings. In the description with reference to the accompanying drawings, identical or corresponding components may be given similar reference numerals, and redundant descriptions thereof may be omitted.

In the disclosure, the term “image” may include medical images obtained by a medical imaging apparatus such as a magnetic resonance imaging (MRI) apparatus, a computed tomography (CT) apparatus, an ultrasound imaging apparatus, or an X-ray imaging apparatus.

In the present disclosure, the term “object” refers to a target to be imaged, and may include a human, an animal, or apart thereof. For example, the term “object” may include apart of a human body (e.g., an organ), a phantom, and the like.

In the disclosure, the term “ultrasound image” refers to an image of an object generated or processed based on ultrasound signals transmitted to and reflected from the object.

Hereinafter, embodiments will be described in detail with reference to the drawings.

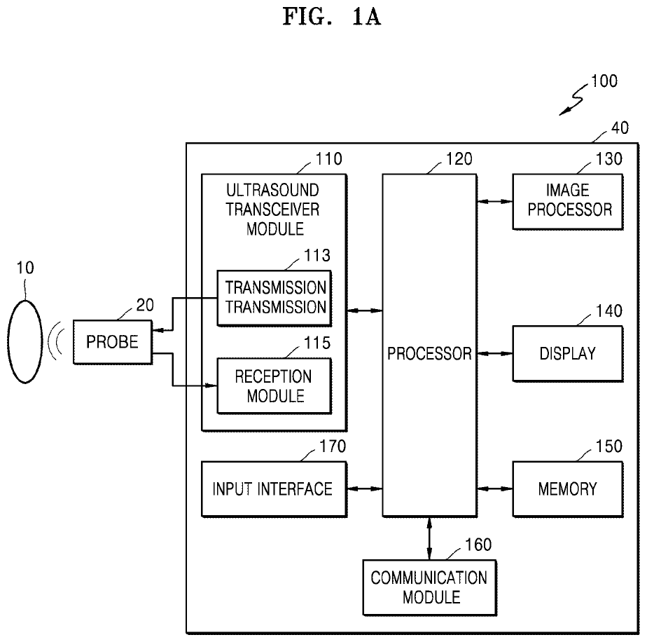

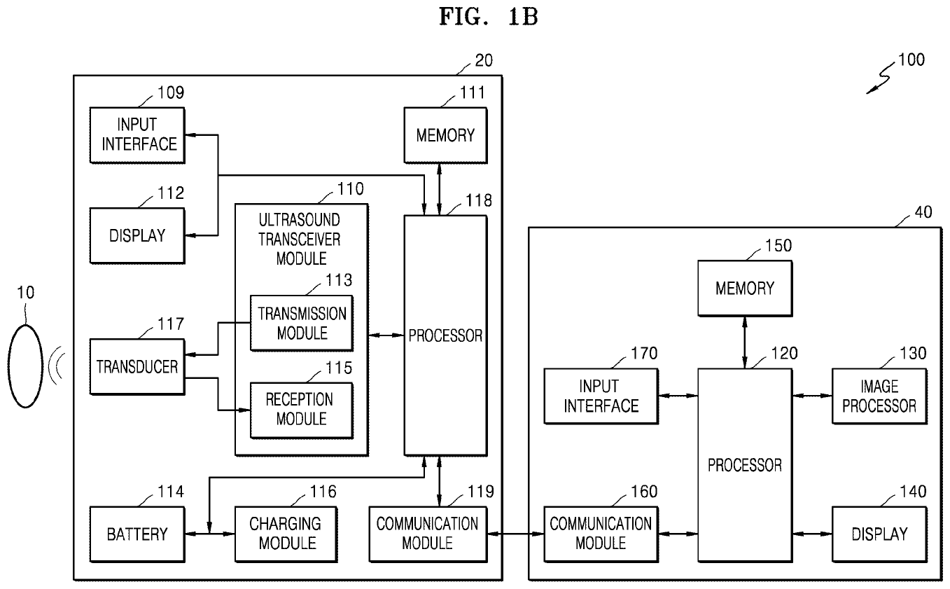

FIG. 1A and FIG. 1B are block diagrams illustrating configurations of an ultrasound imaging system according to an embodiment.

Referring to FIGS. 1A and 1, an ultrasound imaging system 100 may include a probe 20 and an ultrasound imaging apparatus 40.

The ultrasound imaging apparatus 40 may be implemented not only as a cart type but also as a portable type. Examples of portable ultrasound imaging apparatuses may include a smart phone, a laptop computer, a personal digital assistant (PDA), or a tablet personal computer (PC) including a probe and an application, but are not limited thereto. The ultrasound imaging apparatus 40 may also be implemented as a probe-integrated type.

The probe 20 may include a wired probe that communicates with the ultrasound imaging apparatus 40 via a wired connection, a wireless probe that communicates with the ultrasound imaging apparatus 40 via a wireless connection, and/or a hybrid probe that communicates with the ultrasound imaging apparatus 40 via a wired or wireless connection.

According to various embodiments, the ultrasound imaging apparatus 40 may include an ultrasound transceiver module 110 as illustrated in FIG. 1A, or the probe 20 may include the ultrasound transceiver module 110 as illustrated in FIG. 1B. According to various embodiments, both the ultrasound imaging apparatus 40 and the probe 20 may include the ultrasound transceiver module 110.

According to various embodiments, the probe 20 may further include at least one of an image processor 130, a display 140, or an input interface 170, or a combination thereof. In the disclosure, the description of the ultrasound transceiver module 110, the image processor 130, the display 140, or the input interface 170 included in the ultrasound imaging apparatus 40 may also be applied to the ultrasound transceiver module 110, the image processor 130, the display 140, or the input interface 170 included in the probe 20.

FIG. 1A is a block diagram illustrating a configuration of the ultrasound imaging system 100 in a case in which the probe 20 is a wired probe or a hybrid probe.

The probe 20 may include a plurality of transducers. The plurality of transducers may be provided in a certain arrangement to form a transducer array. The transducer array may correspond to a one-dimensional (1D) array or a two-dimensional (2D) array. The plurality of transducers may transmit an ultrasound signal to an object 10 according to a transmission signal applied from a transmission module 113. The plurality of transducers may receive an ultrasound signal (an echo signal) reflected from the object 10 to form a reception signal. In addition, the probe 20 may be implemented integrally with the ultrasound imaging apparatus 40 or as a separate unit connected to the ultrasound imaging apparatus 40 by wire. In addition, depending on the implementation, the ultrasound imaging apparatus 40 may be connected to one or more probes 20.

In a case in which the probe 20 is a wired probe or a hybrid probe, the probe 20 may include a cable and a connector connectable to a connector of the ultrasound imaging apparatus 40.

The probe 20 according to an embodiment may be implemented as a 2D probe. In a case in which the probe 20 is implemented as a 2D probe, the plurality of transducers included in the probe 20 may be arranged two-dimensionally to form a 2D transducer array.

For example, the 2D transducer array may include a plurality of sub-arrays, each including a plurality of transducers arranged in a first direction, wherein the plurality of sub-arrays are arranged in a second direction that is different from the first direction.

In addition, in a case in which the probe 20 according to an embodiment is implemented as a 2D probe, the ultrasound transceiver module 110 may include at least one of an analog beamformer or a digital beamformer. In addition, according to an embodiment, the 2D probe may include at least one of an analog beamformer or a digital beamformer, or a combination thereof, depending on the implementation.

A processor 120 controls the transmission module 113 to generate a transmission signal to be applied to each transducer based on the positions and focal points of the plurality of transducers included in the probe 20.

The processor 120 may control a reception module 115 to perform analog-to-digital conversion on reception signals received from the probe 20, and generate ultrasound data by summing the reception signals that have undergone the analog-to-digital conversion, considering the positions and focal points of the plurality of transducers.

In a case in which the probe 20 is implemented as a 2D probe, the processor 120 may calculate a time delay value for digital beamforming for each of a plurality of sub-arrays included in a 2D transducer array, on a per-sub-array basis. In addition, the processor 120 may calculate a time delay value for analog beamforming for each of transducers included in any one of the plurality of sub-arrays. The processor 120 may control an analog beamformer and a digital beamformer to form transmission signals to be applied to the plurality of transducers, respectively, according to the time delay values for analog beamforming and the time delay values for digital beamforming. In addition, the processor 120 may control the analog beamformer to sum signals received from the plurality of transducers for each sub-array, according to the time delay values for analog beamforming. In addition, the processor 120 may control the ultrasound transceiver module 110 to perform analog-to-digital conversion on a result of summing the signals for each sub-array. In addition, the processor 120 may control the digital beamformer to generate ultrasound data by summing the signals that have undergone the analog-to-digital conversion, according to the time delay values for digital beamforming.

The image processor 130 generates or processes an ultrasound image by using the generated ultrasound data.

The display 140 may display the generated ultrasound image and various pieces of information processed by the ultrasound imaging apparatus 40 or the probe 20. The probe 20 or the ultrasound imaging apparatus 40 may include one or more displays 140 depending on the implementation. In addition, the display 140 may include a touch panel or a touch screen. In addition, the display 140 may include a flexible display.

The processor 120 may control the overall operation of the ultrasound imaging apparatus 40, and may control operations of the components of the ultrasound imaging apparatus 40. The processor 120 may execute programs or instructions stored in a memory 150 to perform or control various operations or functions of the ultrasound imaging apparatus 40. In addition, the processor 120 may receive a control signal from the input interface 170 or an external device, to control an operation of the ultrasound imaging apparatus 40.

The ultrasound imaging apparatus 40 may include a communication module 160, and may connect to and communicate with external devices (e.g., the probe 20, a server, a medical device, or a portable device (e.g., a smart phone, a tablet PC, or a wearable device)) through the communication module 160.

The communication module 160 may include one or more components that enable communication with an external device. The communication module 160 may include, for example, at least one of a short-range communication module, a wired communication module, or a wireless communication module.

The communication module 160 may receive a control signal or data from an external device. The processor 120 may control an operation of the ultrasound imaging apparatus 40 according to a control signal received through the communication module 160. In addition, the processor 120 may transmit a control signal to an external device through the communication module 160 to control the external device according to the transmitted control signal. The external device may operate according to a control signal received from the ultrasound imaging apparatus 40 or may process data received from the ultrasound imaging apparatus 40.

A program or an application associated with the ultrasound imaging apparatus 40 may be installed on the external device. The program or application installed on the external device may control the ultrasound imaging apparatus 40 or may operate according to a control signal or data received from the ultrasound imaging apparatus 40.

The external device may receive or download a program or an application associated with the ultrasound imaging apparatus 40 from the ultrasound imaging apparatus 40, the probe 20, or a server, and install and execute the program or application on the external device. The ultrasound imaging apparatus 40, the probe 20, or the server providing the program or application may include a recording medium storing instructions, commands, installation files, executable files, data, or the like associated with the program or application. The external device may also be sold with the program or application pre-installed.

The memory 150 may store various pieces of data or programs for driving and controlling the ultrasound imaging apparatus 40, input/output ultrasound data, ultrasound images, and the like.

The input interface 170 may receive a user input for controlling the ultrasound imaging apparatus 40. For example, the user input may include, but is not limited to, an input of manipulating a button, a keypad, a mouse, a trackball, a jog switch, a knob, or the like, an input of touching a touch pad or a touch screen, a voice input, a motion input, a biometric information input (e.g., iris recognition or fingerprint recognition), and the like.

FIG. 1B is a control block diagram of the ultrasound imaging system 100 in a case in which the probe 20 is a wireless probe or a hybrid probe.

According to various embodiments, the ultrasound imaging apparatus 40 illustrated in FIG. 1B may be replaced with the ultrasound imaging apparatus 40 described above with reference to FIG. 1A.

According to various embodiments, the probe 20 illustrated in FIG. 1A may be replaced with the probe 20 to be described with reference to FIG. 1B.

The probe 20 may include a display 112, the transmission module 113, a battery 114, a transducer 117, a charging module 116, the reception module 115, an input interface 109, a processor 118, and a communication module 119. Although FIG. 1B illustrates that the probe 20 includes both the transmission module 113 and the reception module 115, depending on the implementation, the probe 20 may include only some of the transmission module 113 and the reception module 115, and some of the transmission module 113 and the reception module 115 may be included in the ultrasound imaging apparatus 40. In addition, according to an embodiment, the probe 20 may further include the image processor 130.

The transducer 117 may include a plurality of transducers. The plurality of transducers may be provided in a certain arrangement to form a transducer array. The transducer array may correspond to a 1D array or a 2D array. The plurality of transducers may transmit an ultrasound signal to an object 10 according to a transmission signal applied from the transmission module 113. In addition, the plurality of transducers may receive an ultrasound signal reflected from the object 10 to form or generate an electrical reception signal.

The charging module 116 may charge the battery 114. The charging module 116 may receive power from an external source. According to an embodiment, the charging module 116 may receive power wirelessly. In addition, according to an embodiment, the charging module 116 may also receive power via a wired connection. The charging module 116 may deliver the received power to the battery 114.

The processor 118 controls the transmission module 113 to generate or form a transmission signal to be applied to each of the plurality of transducers, considering the positions and focal points of the plurality of transducers.

The processor 118 controls the reception module 115 to perform analog-to-digital conversion on a reception signal received from the transducer 117, and to generate ultrasound data by summing reception signals that have undergone the analog-to-digital conversion, considering the positions and focal points of the plurality of transducers. According to an embodiment, in a case in which the probe 20 includes the image processor 130, an ultrasound image may be generated by using the generated ultrasound data.

In a case in which the probe 20 is implemented as a 2D probe, the processor 118 may calculate a time delay value for digital beamforming for each of a plurality of sub-arrays included in a 2D transducer array, on a per-sub-array basis. In addition, the processor 118 may calculate a time delay value for analog beamforming for each of transducers included in any one of the plurality of sub-arrays. The processor 118 may control an analog beamformer and a digital beamformer to form transmission signals to be applied to the plurality of transducers, respectively, according to the time delay values for analog beamforming and the time delay values for digital beamforming. In addition, the processor 118 may control the analog beamformer to sum signals received from the plurality of transducers for each sub-array, according to the time delay values for analog beamforming. In addition, the processor 118 may control the ultrasound transceiver module 110 to perform analog-to-digital conversion on a result of summing the signals for each sub-array. In addition, the processor 118 may control the digital beamformer to generate ultrasound data by summing the signals that have undergone the analog-to-digital conversion, according to the time delay values for digital beamforming.

The processor 118 may control the overall operation of the probe 20, and may control operations of the components of the probe 20. The processor 118 may execute programs or instructions stored in a memory 111 to perform or control various operations or functions of the probe 20. In addition, the processor 118 may receive a control signal from the input interface 109 of the probe 20 or from an external device (e.g., the ultrasound imaging apparatus 40) to control an operation of the probe 20. In addition, the processor 118 may receive a control signal from the input interface 109 or an external device to control an operation of the probe 20. The input interface 109 may receive a user input for controlling the probe 20. For example, the user input may include, but is not limited to, an input of manipulating a button, a keypad, a mouse, a trackball, a jog switch, a knob, or the like, an input of touching a touch pad or a touch screen, a voice input, a motion input, a biometric information input (e.g., iris recognition or fingerprint recognition), and the like.

The display 112 may display an ultrasound image generated by the probe 20, an ultrasound image generated by processing ultrasound data generated by the probe 20, an ultrasound image received from the ultrasound imaging apparatus 40, various pieces of information processed by the ultrasound imaging system 100, or the like. In addition, the display 112 may further display status information about the probe 20. The status information about the probe 20 may include at least one of device information about the probe 20, battery status information about the probe 20, frequency band information about the probe 20, output information about the probe 20, information about the presence or absence of an abnormality of the probe 20, setting information about the probe 20, or temperature information about the probe 20.

The probe 20 may include one or more displays 112 depending on the implementation. In addition, the display 112 may include a touch panel or a touch screen. In addition, the display 112 may include a flexible display.

The communication module 119 may wirelessly transmit generated ultrasound data or ultrasound image to the ultrasound imaging apparatus 40 via a wireless network. In addition, the communication module 119 may receive a control signal and data from the ultrasound imaging apparatus 40.

The ultrasound imaging apparatus 40 may receive ultrasound data or an ultrasound image from the probe 20.

In an embodiment, in a case in which the probe 20 includes the image processor 130 capable of generating an ultrasound image by using ultrasound data, the probe 20 may transmit ultrasound data or an ultrasound image generated by the image processor 130 to the ultrasound imaging apparatus 40.

In an embodiment, in a case in which the probe 20 does not include the image processor 130 capable of generating an ultrasound image by using ultrasound data, the probe 20 may transmit ultrasound data to the ultrasound imaging apparatus 40. Ultrasound data may include raw ultrasound data, and an ultrasound image may refer to ultrasound image data.

The ultrasound imaging apparatus 40 may include the processor 120, the image processor 130, the display 140, the memory 150, the communication module 160, and the input interface 170.

The image processor 130 generates or processes an ultrasound image by using ultrasound data received from the probe 20.

The display 140 may display an ultrasound image received from the probe 20, an ultrasound image generated by processing ultrasound data received from the probe 20, various pieces of information processed by the ultrasound imaging system 100, or the like. The ultrasound imaging apparatus 40 may include one or more displays 140 depending on the implementation. In addition, the display 140 may include a touch panel or a touch screen. In addition, the display 140 may include a flexible display.

The processor 120 may control the overall operation of the ultrasound imaging apparatus 40, and may control operations of the components of the ultrasound imaging apparatus 40. The processor 120 may execute a program or an application stored in the memory 150 to perform or control various operations or functions of the ultrasound imaging apparatus 40. In addition, the processor 120 may receive a control signal from the input interface 170 or an external device, to control an operation of the ultrasound imaging apparatus 40.

The ultrasound imaging apparatus 40 may include the communication module 160, and may connect to and communicate with external devices (e.g., the probe 20, a server, a medical device, or a portable device (e.g., a smart phone, a tablet PC, or a wearable device)) through the communication module 160.

The communication module 160 may include one or more components that enable communication with an external device. The communication module 160 may include, for example, at least one of a short-range communication module, a wired communication module, or a wireless communication module.

The communication module 160 of the ultrasound imaging apparatus 40 and the communication module 119 of the probe 20 may perform communication by using a network, and may perform communication by using a short-range wireless communication method. For example, the communication module 160 of the ultrasound imaging apparatus 40 and the communication module 119 of the probe 20 may perform communication by using any one of wireless data communication methods, including wireless local area network (LAN), Wi-Fi, Bluetooth, Zigbee, Wi-Fi Direct (WFD), Infrared Data Association (IrDA), Bluetooth Low Energy (BLE), near-field communication (NFC), Wireless Broadband Internet (WiBro), Worldwide Interoperability for Microwave Access (WiMAX), Shared Wireless Access Protocol (SWAP), Wireless Gigabit Alliance (WiGig), radio-frequency (RF) communication, or 60-GHz millimeter wave (mmWave) short-range communication.

To this end, the communication module 160 of the ultrasound imaging apparatus 40 and the communication module 119 of the probe 20 may include at least one of a wireless LAN communication module, a Wi-Fi communication module, a Bluetooth communication module, a Zigbee communication module, a WFD communication module, an IrDA module, a BLE communication module, an NFC communication module, a WiBro communication module, a WiMAX communication module, a SWAP communication module, a WiGig communication module, an RF communication module, or a 60-GHz millimeter wave (mmWave) short-range communication module.

In an embodiment, the probe 20 may transmit device information (e.g., identifier (ID) information) about the probe 20 to the ultrasound imaging apparatus 40 by using a first communication method (e.g., BLE), and be wirelessly paired with the ultrasound imaging apparatus 40. In addition, the probe 20 may transmit ultrasound data and/or an ultrasound image to the paired ultrasound imaging apparatus 40.

The device information about the probe 20 may include various pieces of information associated with a serial number, a model name, or battery status of the probe 20.

The ultrasound imaging apparatus 40 may receive device information (e.g., ID information) about the probe 20 from the probe 20 by using the first communication method (e.g., BLE), and be wirelessly paired with the probe 20. In addition, the ultrasound imaging apparatus 40 may transmit an activation signal to the paired probe 20 and receive ultrasound data and/or an ultrasound image from the probe 20. Here, the activation signal may include a signal for controlling an operation of the probe 20.

In an embodiment, the probe 20 may transmit device information (e.g., ID information) about the probe 20 to the ultrasound imaging apparatus 40 by using the first communication method (e.g., BLE), and be wirelessly paired with the ultrasound imaging apparatus 40. In addition, by using a second communication method (e.g., 60-GHz millimeter wave or Wi-Fi), the probe 20 may transmit ultrasound data and/or an ultrasound image to the ultrasound imaging apparatus 40, which is paired with the probe 20 by using the first communication method.

The ultrasound imaging apparatus 40 may receive device information (e.g., ID information) about the probe 20 from the probe 20 by using the first communication method (e.g., BLE), and be wirelessly paired with the probe 20. In addition, the ultrasound imaging apparatus 40 may transmit an activation signal to the paired probe 20 and receive ultrasound data and/or an ultrasound image from the probe 20 by using the second communication method (e.g., 60-GHz millimeter wave or Wi-Fi).

According to an embodiment, the first communication method used for pairing the probe 20 with the ultrasound imaging apparatus 40 may have a lower frequency band than that of the second communication method used for the probe 20 to transmit ultrasound data and/or an ultrasound image to the ultrasound imaging apparatus 40.

The display 140 of the ultrasound imaging apparatus 40 may display user interfaces (UIs) indicating device information about the probe 20. For example, the display 140 may display UIs indicating identification information about the wireless ultrasound probe 20, a method of pairing with the probe 20, the data communication status between the probe 20 and the ultrasound imaging apparatus 40, a method of performing data communication with the ultrasound imaging apparatus 40, the battery status of the probe 20, or the like.

In a case in which the probe 20 includes the display 112, the display 112 of the probe 20 may display a UI indicating device information about the probe 20. For example, the display 112 may display UIs indicating identification information about the wireless ultrasound probe 20, a method of pairing with the probe 20, the data communication status between the probe 20 and the ultrasound imaging apparatus 40, a method of performing data communication with the ultrasound imaging apparatus 40, the battery status of the probe 20, or the like.

The communication module 160 may receive a control signal or data from an external device. The processor 120 may control an operation of the ultrasound imaging apparatus 40 according to a control signal received through the communication module 160.

In addition, the processor 120 may transmit a control signal to an external device through the communication module 160 to control the external device according to the transmitted control signal. The external device may operate according to a control signal received from the ultrasound imaging apparatus 40 or may process data received from the ultrasound imaging apparatus 40.

The external device may receive or download a program or an application associated with the ultrasound imaging apparatus 40 from the ultrasound imaging apparatus 40, the probe 20, or a server, and install and execute the program or application on the external device. The ultrasound imaging apparatus 40, the probe 20, or the server providing the program or application may include a recording medium storing instructions, commands, installation files, executable files, data, or the like associated with the program or application. The external device may also be sold with the program or application pre-installed.

The memory 150 may store various pieces of data or programs for driving and controlling the ultrasound imaging apparatus 40, input/output ultrasound data, ultrasound images, and the like.

The processor 120 may include at least one processor.

The at least one processor 120 may include processing circuitry.

The memory 150 may store instructions.

When the instructions are executed by the at least one processor 120 individually or collectively, the ultrasound imaging apparatus 40 may perform at least one function or embodiment.

The at least one processor 120 may receive, via the input interface 170, a user input for selecting a cardiac imaging option. The at least one processor 120 may receive, via the input interface 170, a user input for selecting a fetal cardiac imaging option.

Based on a user input, the at least one processor 120 may emit, via the ultrasound transceiver module 110, ultrasound signals toward the heart of an object, and obtain ultrasound images of a cycle of the heart based on ultrasound echo signals received from the object.

The orientation of the heart in the ultrasound images is not fixed and may appear in any direction. For example, the ultrasound images include two or more ultrasound images in which orientations of the heart differ by at least a reference angle. For example, the reference angle may be a value between 90 degrees and 180 degrees.

The at least one processor 120 may receive, via the input interface 170, a user input for selecting an option to check cardiac information from the obtained ultrasound images.

Based on a user input, the at least one processor 120 may identify regions of a plurality of chambers in the obtained ultrasound images.

The at least one processor 120 may determine the position of the apex of a first chamber and the position of the valve of the first chamber based on a relative position between the apex and valve of the first chamber and a second chamber, among the plurality of chambers. For example, the at least one processor 120 may determine, as the position of the apex of the left ventricle, the point in the region of the left ventricle that is farthest from the left atrium. The at least one processor 120 may determine, as the position of the valve, the point in the region of the left ventricle that is closest to the right atrium.

The at least one processor 120 may calculate information about the heart in the cardiac cycle based on the position of the apex and the position of the valve.

The at least one processor 120 may display the information about the heart via the display 140.

The at least one processor 120 may display the positions of the apex and the valve of the first chamber on the ultrasound images via the display 140.

The at least one processor 120 may receive, via the input interface 170, a user input for selecting one of the displayed apex and valve. The at least one processor 120 may receive, via the input interface 170, a user input for adjusting the position of the selected apex or valve. The at least one processor 120 may recalculate the information about the heart based on the adjusted position of the apex or valve.

The at least one processor 120 may display, via the display 140, a graph showing the information about the heart during a plurality of cycles.

The at least one processor 120 may determine an optimal cycle from among the plurality of cycles of the heart. The at least one processor 120 may display, via the display 140, the determined optimal cycle on the graph showing the information about the heart during the plurality of cycles.

The at least one processor 120 may identify regions of the first chamber, which include a contour of the valve of the first chamber, in the obtained ultrasound images.

The at least one processor 120 may display, via the display 140, a contour of the first chamber on the ultrasound images. The at least one processor 120 may display, via the display 140, the contour of the valve of the first chamber to be distinguishable from a contour of the inner wall of the first chamber.

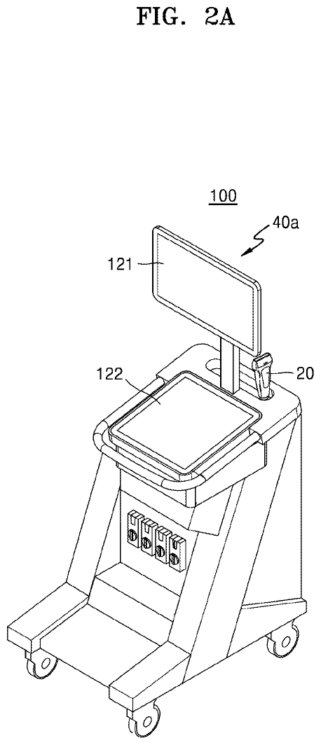

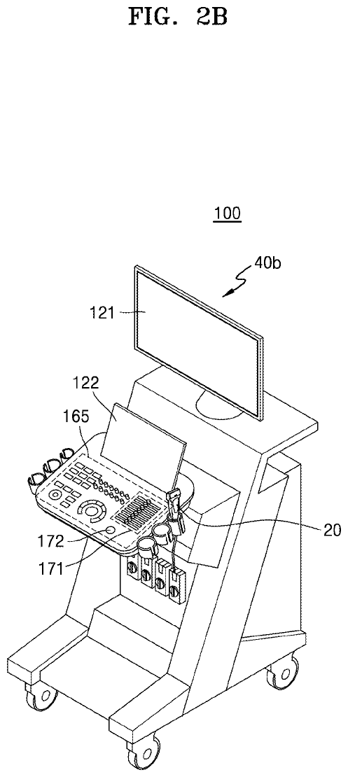

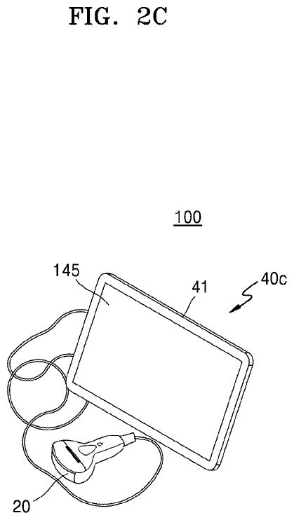

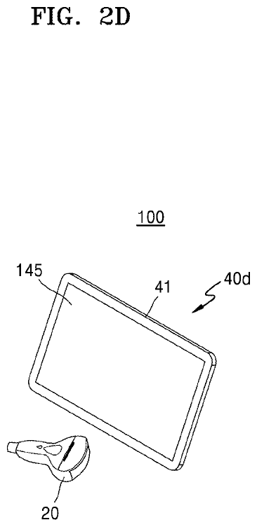

Examples of the ultrasound imaging system 100 according to an embodiment will be described below with reference to FIGS. 2A, 2B, 2C, and 2D.

FIGS. 2A, 2B, 2C, and 2D are diagrams each illustrating an ultrasound imaging apparatus according to an embodiment.

Referring to FIGS. 2A and 2B, ultrasound imaging apparatuses 40a and 40b may each include a main display 121 and a sub-display 122. The main display 121 and the sub-display 122 may correspond to the display 140 of FIGS. 1A and 1B. At least one of the main display 121 or the sub-display 122 may be implemented as a touch screen. At least one of the main display 121 or the sub-display 122 may display an ultrasound image or various pieces of information processed by the ultrasound imaging apparatus 40a or 40b. In addition, at least one of the main display 121 or the sub-display 122 may be implemented as a touch screen, and may provide a graphical user interface (GUI) to receive, from a user, data for controlling the ultrasound imaging apparatus 40a or 40b. For example, the main display 121 may display an ultrasound image, and the sub-display 122 may display a control panel in a GUI form for controlling the display of the ultrasound image. The sub-display 122 may receive data for controlling the display of the image, through the control panel displayed in the GUI form. For example, a time gain compensation (TGC) button, a lateral gain compensation (LGC) button, a Freeze button, a trackball, a jog switch, a knob, or the like may be provided as a GUI on the sub-display 122.

The ultrasound imaging apparatuses 40a and 40b may control the display of the ultrasound image on the main display 121 by using control data that is received as an input. In addition, the ultrasound imaging apparatuses 40a and 40b may be connected to the probe 20 by wire or wirelessly to transmit and receive ultrasound signals to and from an object.

Referring to FIG. 2B, the ultrasound imaging apparatus 40b may further include a control panel 165 in addition to the main display 121 and the sub-display 122. The control panel 165 may include buttons, a trackball, a jog switch, a knob, and the like, and may receive, from the user, data for controlling the ultrasound imaging apparatus 40b. For example, the control panel 165 may include a TGC button 171, a Freeze button 172, and the like. The TGC button 171 is for setting a TGC value for each depth of an ultrasound image. In addition, when an input from the Freeze button 172 is detected while scanning an ultrasound image, the ultrasound imaging apparatus 40b may maintain the display of the frame image at the corresponding time point, capture the frame image at the corresponding time point, or store the frame image at the corresponding time point.

In addition, the buttons, trackball, jog switch, knob, and the like included in the control panel 165 may be provided as GUIs on the main display 121 or the sub-display 122. In addition, the ultrasound imaging apparatuses 40a and 40b may be connected to the probe 20 to transmit and receive ultrasound signals to and from an object.

In addition, the ultrasound imaging apparatuses 40a and 40b may include various types of input/output interfaces such as a speaker, a light-emitting diode (LED), or a vibration device. For example, the ultrasound imaging apparatuses 40a and 40b may output various pieces of information in the form of graphics, sound, or vibration, through the input/output interfaces. In addition, the ultrasound imaging apparatuses 40a and 40b may output various notifications or various pieces of data through the input/output interfaces.

Referring to FIGS. 2C and 2D, ultrasound imaging apparatuses 40c and 40d may be implemented as a portable type. Examples of the portable ultrasound imaging apparatuses 40c and 40d may include a smart phone, a laptop computer, a PDA, or a tablet PC including a probe and an application, but are not limited thereto.

The ultrasound imaging apparatus 40c may include a main body 41. Referring to FIG. 2C, the probe 20 may be connected by wire to one side of the main body 41. To this end, the main body 41 may include a connection terminal to which a cable connected to the probe 20 may be detachably connected. The probe 20 may include a cable including a connection terminal connectable to the main body 41.

Referring to FIG. 2D, the probe 20 may be wirelessly connected to the ultrasound imaging apparatus 40d. The main body 41 may include an input/output interface (e.g., a touch screen). The input/output interface may display an ultrasound image, various pieces of information processed by the ultrasound imaging apparatus 40d, a GUI, or the like.

The ultrasound imaging apparatus 40d and the probe 20 may establish communication or be paired with each other by using short-range wireless communication. For example, the ultrasound imaging apparatus 40d and the probe 20 may perform communication by using Bluetooth, BLE, Wi-Fi, WFD, or the like.

The ultrasound imaging apparatuses 40c and 40d may execute a program or an application associated with the probe 20 to control the probe 20 and to output information associated with the probe 20. The ultrasound imaging apparatuses 40c and 40d may perform operations associated with the probe 20 while communicating with a certain server. The probe 20 may be registered with the ultrasound imaging apparatus 40c or 40d, or with a certain server. The ultrasound imaging apparatuses 40c and 40d may communicate with the registered probe 20 and perform operations associated with the probe 20.

In addition, the ultrasound imaging apparatuses 40c and 40d may include various types of input/output interfaces such as a speaker, an LED, or a vibration device. For example, the ultrasound imaging apparatuses 40c and 40d may output various pieces of information in the form of graphics, sound, or vibration, through the input/output interfaces. In addition, the ultrasound imaging apparatuses 40c and 40d may output various notifications or various pieces of data through the input/output interfaces.

According to an embodiment, the ultrasound imaging apparatus 40a, 40b, 40c, or 40d may process an ultrasound image or obtain additional information from an ultrasound image, by using an artificial intelligence (AI) model. According to an embodiment, the ultrasound imaging apparatus 40a, 40b, 40c, or 40d may generate an ultrasound image or perform processing, such as correction, image quality enhancement, encoding, or decoding, on an ultrasound image, by using an AI model. In addition, according to an embodiment, the ultrasound imaging apparatus 40a, 40b, 40c, or 40d may perform processing, such as defining a baseline, obtaining anatomical information, obtaining lesion information, extracting a surface, defining a boundary, measuring a length, measuring an area, measuring a volume, or generating an annotation, from an ultrasound image by using an AI model.

The AI model may be provided in the ultrasound imaging apparatus 40a, 40b, 40c, or 40d, or a server.

The AI model may be implemented by using various artificial neural network models or deep neural network models. In addition, the AI model may be trained and generated by using various machine learning algorithms or deep learning algorithms. The AI model may be implemented by using models such as a convolutional neural network (CNN), a recurrent neural network (RNN), a generative adversarial network (GAN), or long short-term memory (LSTM).

Embodiments are to provide the position of an apex within a cardiac ultrasound image.

Embodiments are to provide the position of a valve within a cardiac ultrasound image.

Embodiments are to provide information about a heart from a cardiac ultrasound image.

Embodiments are to provide information about a plurality of cardiac cycles.

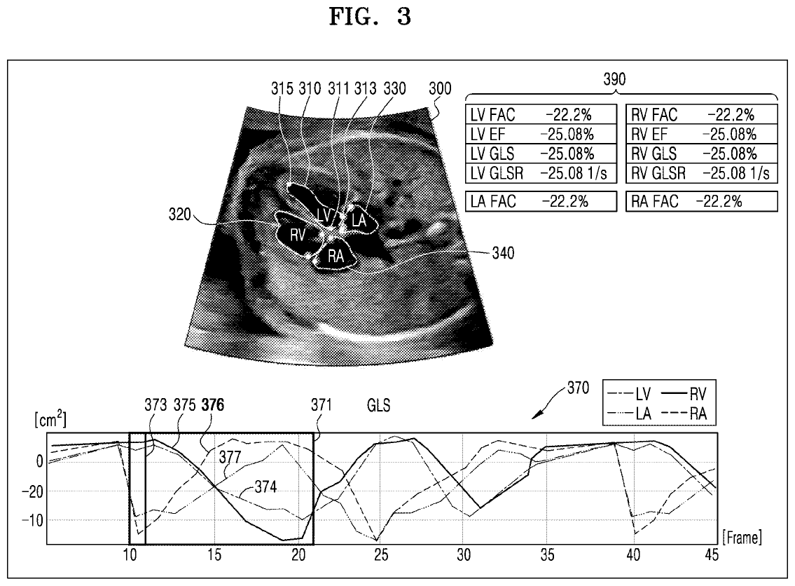

FIG. 3 illustrates a method, performed by the ultrasound imaging apparatus 40, of providing information about a heart, according to an embodiment.

Referring to FIG. 3, the ultrasound imaging apparatus 40 may obtain a plurality of ultrasound images of an object's heart. The ultrasound imaging apparatus 40 may identify regions of chambers in each of the plurality of ultrasound images. The ultrasound imaging apparatus 40 may display the identified regions of a left ventricle 310, a right ventricle 320, a left atrium 330, and a right atrium 340, on an ultrasound image 300.

The ultrasound imaging apparatus 40 may determine the positions of an apex and a valve within a chamber based on a relative position between the apex and valve and other chambers.

For example, the left ventricle 310 has an elongated triangular shape with an apex 315 as its vertex, and start points 311 and 313 of two mitral valves as the vertices of its base. In addition, the left ventricle 310, the right ventricle 320, the left atrium 330, and the right atrium 340 are located facing each other. Accordingly, the apex 315 of the left ventricle 310 may be the point in the region of the left ventricle 310 that is farthest from the left atrium 330. In addition, the medial start point 311 of the mitral valve of the left ventricle 310 may be the point in the region of the left ventricle 310 that is closest to the right atrium 340. Accordingly, the ultrasound imaging apparatus 40 may identify the apex 315 and the start points 311 and 313 of the valves of the left ventricle 310 based on the morphological features of the left ventricle 310 and the relative positions among the chambers.

According to an embodiment, the ultrasound imaging apparatus 40 may also detect the position of an apex and the position of a valve by using an AI model. For example, an AI model may be trained to output, when an ultrasound image is input, the positions of a valve and the apex of a chamber. However, in a case in which an AI model is used, training takes time, and an inaccurate position may be output depending on the training data.

In addition, for a fetal cardiac ultrasound image or an ultrasound image captured in an emergency situation, it may be difficult to detect the accurate positions of an apex and a valve by using an AI model because the angle of the heart is not constant.

In detail, to obtain a 4-chamber view of a heart, a user generally maintains the horizontal orientation of the probe within ±450 relative to the ventricular septum. Accordingly, a cardiac ultrasound image of a general person may be captured such that the apex of a ventricle is located at an uppermost part of the ultrasound image. However, in a case of a fetus, it is difficult to adjust the angle of the probe for the fetal heart due to the fetus's movement and surrounding structures. Thus, the direction of the apex of the ventricle is not fixed in the ultrasound image and may appear in any direction. For example, in a fetal cardiac ultrasound image, the apex of a ventricle may be located at a lower part of the ultrasound image.

According to an embodiment, the ultrasound imaging apparatus 40 may accurately and quickly identify the positions of an apex and a valve of a fetal heart, regardless of the angle of the probe with respect to the fetal heart, by determining the position of the apex and the position of the valve based on a relative position between the apex and valve within the chamber and other chambers.

The ultrasound imaging apparatus 40 may provide information about the heart from the obtained ultrasound images. For example, the ultrasound imaging apparatus 40 may calculate a fractional area change (FAC) of the chamber, an ejection fraction (EF) of the chamber, and a global longitudinal strain rate (GLSR) of the chamber.

In addition, the ultrasound imaging apparatus 40 may calculate a global longitudinal strain (GLS) of the chamber based on the positions of the apex and the valve. In a heart, an apex may be a fixed region that hardly moves during contraction and relaxation of the heart. Thus, the ultrasound imaging apparatus 40 may provide an accurate longitudinal strain by calculating, as the longitudinal strain of the chamber, the rate of change in the length from the apex to the start point of the valve.

The ultrasound imaging apparatus 40 may identify the positions of the apexes and valves of the right ventricle 320, the right atrium 340, and the left atrium 330, as well as the left ventricle 310, and determine the longitudinal strain for each chamber. The ultrasound imaging apparatus 40 may display a graph 370 showing the longitudinal strains according to each frame. In this case, the ultrasound imaging apparatus 40 may obtain the longitudinal strains of the chambers for each of the obtained ultrasound images. The longitudinal strain of a chamber for a single ultrasound image may be the ratio of the longitudinal length of the chamber in the ultrasound image to the longitudinal length of the chamber at the time of the immediately preceding end-diastole.

In addition, the ultrasound imaging apparatus 40 may display a longitudinal strain graph 374 of the left ventricle, a longitudinal strain graph 375 of the right ventricle, a longitudinal strain graph 377 of the left atrium, and a longitudinal strain graph 376 of the right atrium.

The ultrasound imaging apparatus 40 may display the apex and the start point of a valve of each chamber. For example, the ultrasound imaging apparatus 40 may display the detected apex 315 and the detected start points 311 and 313 of the mitral valves on the region of the left ventricle 310 in the ultrasound image 300.

The ultrasound imaging apparatus 40 may identify a systole and a diastole based on a change in the size of the chambers. For example, the ultrasound imaging apparatus 40 may determine, as the diastole, the time point when the left ventricle 310 is largest, and determine, as the systole, the time point when the left atrium 330 is largest. The ultrasound imaging apparatus 40 may determine, as one cycle of the heart, the time period from one diastole to the next diastole.

In addition, the ultrasound imaging apparatus 40 may display an image 371 indicating a focused cycle of the heart among a plurality of cycles of the heart. The ultrasound imaging apparatus 40 may provide cardiac information for the focused cycle of the heart. For example, the ultrasound imaging apparatus 40 may display cardiac function indicator information 390 including a GLS, an FAC, an EF of the chamber, and a GLSR for the focused cycle of the heart.

Accordingly, a user may check information about the heart simply via an input for capturing a cardiac ultrasound image, without user inputs for inputting the contour of the chamber or the positions of valves and apexes on an ultrasound image. In particular, even when it is difficult to adjust the display orientation of valves and apexes, such as in fetal cardiac ultrasound images or emergency cardiac ultrasound images, the ultrasound imaging apparatus 40 may automatically calculate the longitudinal strain of the heart.

According to an embodiment, the ultrasound imaging apparatus 40 may identify not only the inner wall of the chamber but also the contour of a valve of the chamber, and display the identified contour of the valve on the ultrasound image. For example, the ultrasound imaging apparatus 40 may determine a contour of a chamber, in which a contour of a valve is represented, by using an AI model. The ultrasound imaging apparatus 40 may determine, as the contour of the valve, a part between the start points of two valves in the contour of the chamber. Accordingly, the user may check not only the movement of the chamber but also the movement of the valve from the ultrasound images.

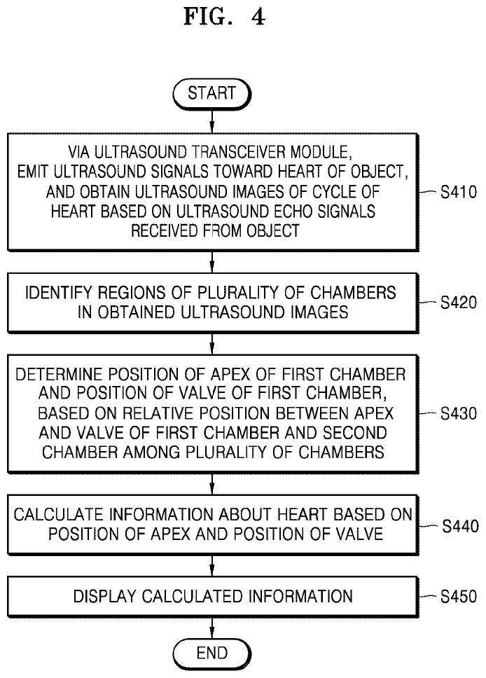

FIG. 4 is a flowchart of a method, performed by the ultrasound imaging apparatus 40, of calculating information about a heart from cardiac ultrasound images, according to an embodiment.

In operation S410, the ultrasound imaging apparatus 40 may, via the ultrasound transceiver module, emit ultrasound signals toward the heart of an object, and obtain ultrasound images of a cycle of the heart based on ultrasound echo signals received from the object.

The ultrasound imaging apparatus 40 may receive a user input for selecting a cardiac imaging option. As the probe is brought into contact with the vicinity of an apical region of the object's heart, the ultrasound imaging apparatus 40 may obtain cardiac ultrasound images of the object.

The ultrasound imaging apparatus 40 may receive a user input for selecting a fetal cardiac imaging option, and obtain cardiac ultrasound images of a fetus. In a case of fetal cardiac ultrasound imaging, it may not be easy to capture an image such that the apex of a ventricle is located at an upper part of the ultrasound image, because it is difficult to adjust the angle of the probe relative to the fetal heart. Accordingly, the direction of the apex of the ventricle within the obtained ultrasound images may not be fixed.

In operation S420, the ultrasound imaging apparatus 40 may identify regions of a plurality of chambers in the obtained ultrasound images.

The ultrasound imaging apparatus 40 may receive a user input for selecting an option to check cardiac information from the obtained ultrasound images. Based on the user input for selecting the option to check cardiac information from the ultrasound images, the ultrasound imaging apparatus 40 may identify regions of a plurality of chambers in the obtained ultrasound images.

The ultrasound imaging apparatus 40 may segment the regions of the left ventricle, the right ventricle, the left atrium, and the right atrium in the obtained ultrasound images by using an AI model.

In operation S430, the ultrasound imaging apparatus 40 may determine the position of the apex of a first chamber and the position of a valve of the first chamber, based on a relative position between the apex and valve of the first chamber and a second chamber among the plurality of chambers.

For example, the ultrasound imaging apparatus 40 may determine, as the position of the apex of the left ventricle, the point in the region of the left ventricle that is farthest from the left atrium. In addition, for example, the ultrasound imaging apparatus 40 may determine, as the position of the valve, the point in the region of the left ventricle that is closest to the right atrium.

In operation S440, the ultrasound imaging apparatus 40 may calculate information about the heart based on the position of the apex and the position of the valve.

The ultrasound imaging apparatus 40 may determine the cycle of the heart based on the areas of the chambers. For example, the ultrasound imaging apparatus 40 may generate a graph of the area of the left ventricle according to each frame, and determine, as the end-diastole of the heart, the time points at which the area of the left ventricle shows a peak value. In addition, the ultrasound imaging apparatus 40 may generate a graph of the area of the left atrium according to each frame, and determine, as the end-systole of the heart, the time points at which the area of the left atrium shows a peak value. The ultrasound imaging apparatus 40 may determine, as one cycle of the heart, the period from one end-diastole to the next end-diastole.

When ultrasound images are obtained during a plurality of cycles of the heart, the ultrasound imaging apparatus 40 may determine the plurality of cycles of the heart based on the areas of the chambers.

The ultrasound imaging apparatus 40 may calculate cardiac information from the obtained ultrasound images. For example, the ultrasound imaging apparatus 40 may calculate a GLS of the cycle of the heart. In this case, the ultrasound imaging apparatus 40 may calculate, as the longitudinal strain of the heart, the ratio between the length from the apex to the valve at the time of end-diastole and the length from the apex to the valve at the time of end-systole.

In addition, for example, the ultrasound imaging apparatus 40 may calculate an FAC of the chamber, an EF of the chamber, and a GLSR of the chamber, which correspond to the cycle of the heart, but is not limited thereto.

In operation S450, the ultrasound imaging apparatus 40 may display the calculated information.

For example, the ultrasound imaging apparatus 40 may display the calculated GLS, FAC, EF, or GLSR of the chamber.

According to an embodiment, the ultrasound imaging apparatus 40 may display the positions of the apex and the valve of the chamber on the ultrasound images.

According to an embodiment, the ultrasound imaging apparatus 40 may receive a user input for selecting one of the displayed apex or valve and adjusting the position of the selected apex or valve. The ultrasound imaging apparatus 40 may recalculate the information about the heart based on the adjusted position of the apex or valve.

According to an embodiment, the ultrasound imaging apparatus 40 may identify a plurality of cycles of the heart from the obtained ultrasound images. The ultrasound imaging apparatus 40 may calculate information about the heart during the plurality of cycles based on the positions of apexes and the positions of valves. The ultrasound imaging apparatus 40 may display a graph showing information about the heart during the plurality of cycles.

According to an embodiment, the ultrasound imaging apparatus 40 may recommend an optimal cycle among the plurality of cycles of the heart. For example, the ultrasound imaging apparatus 40 may determine, as the optimal cycle, the cycle having the largest amount of change in longitudinal strain among the plurality of cycles of the heart.

According to an embodiment, the ultrasound imaging apparatus 40 may display the determined optimal cycle on the graph showing the information about the heart during the plurality of cycles.

According to an embodiment, the ultrasound imaging apparatus 40 may display a contour of the chamber on the ultrasound image such that the contour of the valve of the chamber is distinguishable from the contour of the inner wall of the chamber.

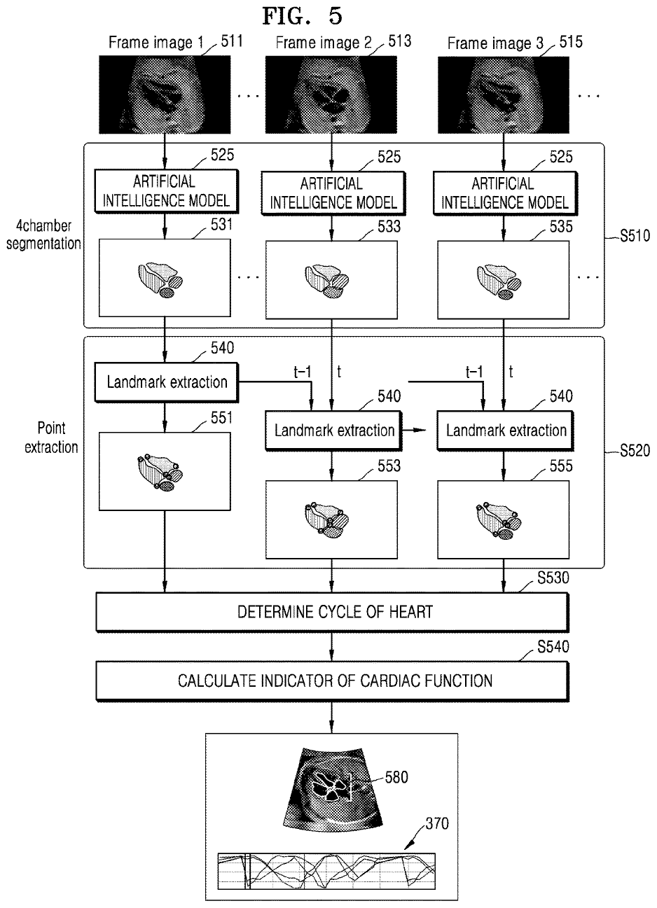

FIG. 5 illustrates a flowchart of a method, performed by the ultrasound imaging apparatus 40, of calculating information about a heart from cardiac ultrasound images, according to an embodiment.

In operation S510, the ultrasound imaging apparatus 40 may segment regions of chambers in cardiac ultrasound images.

The ultrasound imaging apparatus 40 may receive a user input for capturing the heart of an object by using the probe. For example, in response to receiving a user input for capturing for 5 seconds at 70 fps after bringing the probe into contact with an area near the apex of the object's heart, the ultrasound imaging apparatus 40 may obtain 350 frames of B-mode ultrasound images in a 4-chamber view. The ultrasound imaging apparatus 40 may also obtain B-mode ultrasound images in a 2-chamber view, depending on the angle of the probe relative to the heart.

The ultrasound imaging apparatus 40 may also obtain previously captured cardiac ultrasound images from a server or an external device.

According to an embodiment, the ultrasound imaging apparatus 40 may also perform intensity normalization on B-mode ultrasound images.

The ultrasound imaging apparatus 40 may identify regions of four chambers (left ventricle, right ventricle, left atrium, and right atrium) from ultrasound images 511 to 515. For example, the ultrasound imaging apparatus 40 may detect the four chamber regions by using an AI model 525 that outputs the regions of the left ventricle, the right ventricle, the left atrium, and the right atrium when an ultrasound image is input.

The AI model 525 may be a model that has been trained based on supervised learning to output the positions of the regions of the left ventricle, the right ventricle, the left atrium, and the right atrium when an ultrasound image in a 4-chamber view is input. To this end, ultrasound images in a 4-chamber view may be set as input data for training data, and images in a 4-chamber view in which the regions of the left ventricle, the right ventricle, the left atrium, and the right atrium are labeled may be set as target data for the training data.

By training the AI model 525 based on a sufficient amount of training data, parameters of the AI model 525, such as weights of a neural network, may be determined. Accordingly, the AI model 525 may output an image 531 indicating the positions of regions of the left ventricle, the right ventricle, the left atrium, and the right atrium when an ultrasound image 511 in a 4-chamber view is input. For example, as illustrated in FIG. 5, the AI model 525 may output the image 531 indicating the region of the left ventricle in red, the region of the right ventricle in green, the region of the left atrium in yellow, and the region of the right atrium in blue. Accordingly, the ultrasound imaging apparatus 40 may determine the region of the ultrasound image 511 corresponding to one chamber in the image 531, as the region of the chamber.

The ultrasound imaging apparatus 40 may obtain, via the AI model 525, an image 531, 533, or 535, which indicates the positions of the regions of the left ventricle, the right ventricle, the left atrium, and the right atrium, for each of a plurality of ultrasound images 511 to 515 in a 4-chamber view.

FIG. 5 illustrates the ultrasound images 511 to 515 in a 4-chamber view as an example, but according to an embodiment, the ultrasound images may be in a 2-chamber view. In this case, the AI model may output an image indicating the regions of the left ventricle and the left atrium in an ultrasound image in a 2-chamber view when the ultrasound image is input. An AI model for a 4-chamber view and an AI model for a 2-chamber view may be provided together in the ultrasound imaging apparatus 40.

According to an embodiment, the AI model 525 may output a contour of the chamber such that a contour of a valve is revealed. The ultrasound imaging apparatus 40 may represent the region of the chamber more accurately by identifying a region of the chamber in which the movement of the valve is reflected.

In operation S520, the ultrasound imaging apparatus 40 may identify the positions of the apex and the valve on the identified region of the chamber.

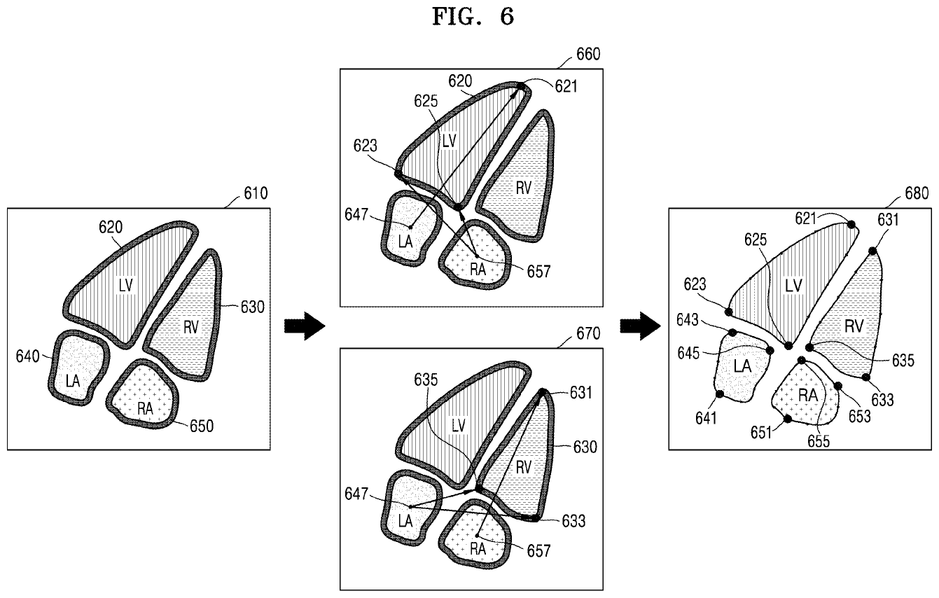

The ultrasound imaging apparatus 40 may detect the position of the apex and the start point of a valve for each of the left ventricle, the right ventricle, the left atrium, and the right atrium, through a landmark extraction operation 540. For example, the ultrasound imaging apparatus 40 may determine the positions of an apex and a valve within a chamber based on a relative position between the apex and valve and other chambers. A method of determining the positions of an apex and a valve of a chamber will be described in detail with reference to FIGS. 6 to 8.

For each of the images 531, 533, and 535 indicating the regions of the four chambers, the ultrasound imaging apparatus 40 may determine the apex and the start point of the valve for each of the four chambers. In addition, the ultrasound imaging apparatus 40 may obtain an image 551, 553, or 555 in which the positions of the determined apex and start point of the valve are indicated.

In operation S530, the ultrasound imaging apparatus 40 may determine the cycle of the heart based on the size of the identified region of the chamber.

The ultrasound imaging apparatus 40 may determine, as one cycle, the period from the end-diastole of the heart, through the systole, to the next end-diastole. The ultrasound imaging apparatus 40 may determine, as the end-diastole of the heart, the time point when the area of the left ventricle region is largest, and determine, as the end-systole, the time point when the area of the left atrium region is largest.

When ultrasound images are obtained during a plurality of cycles of the heart, the ultrasound imaging apparatus 40 may determine, as the end-diastole of the heart, the time points at which the area of the left ventricle region shows a peak value within a reference time period. The reference time period may be predetermined based on the general heart rate of the object. The ultrasound imaging apparatus 40 may determine, as the end-systole of the heart, the time point at which the area of the left atrium region shows a peak value, within the period between adjacent end-diastoles.

According to an embodiment, the ultrasound imaging apparatus 40 may determine the diastole and the systole more accurately by using the area of the chamber that reflects the contour of the valve.

In operation S540, the ultrasound imaging apparatus 40 may calculate an indicator of the cardiac function of the object.

For example, the ultrasound imaging apparatus 40 may calculate a rate of change of the area for each of the chambers. The ultrasound imaging apparatus 40 may calculate a longitudinal strain, which is the rate of change of the length from the apex to the valve of each chamber.

The ultrasound imaging apparatus 40 may display an image 580 showing the identified regions of the chambers on the ultrasound image. In addition, the ultrasound imaging apparatus 40 may display a graph showing the rates of change of the areas of the chambers over time. In addition, the ultrasound imaging apparatus 40 may display the graph 370 showing the rates of change of the longitudinal lengths of the chambers over time. In addition, the ultrasound imaging apparatus 40 may display the calculated indicator of the cardiac function.

FIG. 6 illustrates a method, performed by the ultrasound imaging apparatus 40, of determining the positions of the apex and a valve of a chamber, according to an embodiment.

Referring to FIG. 6, the ultrasound imaging apparatus 40 may determine the positions of an apex and a valve within a chamber based on a relative position between the apex and valve and other chambers.

The ultrasound imaging apparatus 40 may identify the regions of the left ventricle, the right ventricle, the left atrium, and the right atrium from an ultrasound image. The ultrasound imaging apparatus 40 may obtain a chamber segmentation image 610 that distinguishably indicates the identified regions of the chambers.

For example, in the chamber segmentation image 610, a left ventricle region 620 may be displayed with a red mask, a right ventricle region 630 may be displayed with a green mask, a left atrium region 640 may be displayed with a yellow mask, and a right atrium region 650 may be displayed with a blue mask.

The ultrasound imaging apparatus 40 may determine a contour of each of the chamber regions 620, 630, 640, and 650 on the chamber segmentation image 610.

The ultrasound imaging apparatus 40 may determine the apex and the start point of a valve of the chamber on the chamber segmentation image 610, based on a relative position between the apex and valve within the chamber and other chambers.

Referring to the top center image (660), the ultrasound imaging apparatus 40 may determine an apex 621, a start point 625 of a first mitral valve, and a start point 623 of a second mitral valve in the left ventricle region.

For example, the ultrasound imaging apparatus 40 may determine a center 647 of the left atrium region and a center 657 of the right atrium region. The ultrasound imaging apparatus 40 may determine, as the apex 621 of the left ventricle, the point on the contour of the left ventricle region 620 that is farthest from the center 647 of the left atrium region. In addition, the ultrasound imaging apparatus 40 may determine, as the start point 625 of the first mitral valve of the left ventricle, the point on the contour of the left ventricle region 620 that is closest to the center 657 of the right atrium region. In addition, the ultrasound imaging apparatus 40 may determine, as the start point 623 of the second mitral valve, the point on the contour of the left ventricle region 620 that is farthest counterclockwise from the center 657 of the right atrium region, relative to the start point 625 of the first mitral valve.

Referring to the bottom center image (670), the ultrasound imaging apparatus 40 may determine an apex 631, a start point 635 of a first tricuspid valve, and a start point 633 of a second tricuspid valve in the right ventricle region.

For example, the ultrasound imaging apparatus 40 may determine, as the apex 631 of the right ventricle, the point on the contour of the right ventricle region 630 that is farthest from the center 657 of the right atrium region. In addition, the ultrasound imaging apparatus 40 may determine, as the start point 635 of the first tricuspid valve of the right ventricle, the point on the contour of the right ventricle region 630 that is closest to the center 647 of the left atrium region. In addition, the ultrasound imaging apparatus 40 may determine, as the start point 633 of the second tricuspid valve, the point on the contour of the right ventricle region 630 that is farthest clockwise from the center 647 of the left atrium region, relative to the start point 635 of the first tricuspid valve.

Referring to the right image (680), the ultrasound imaging apparatus 40 may determine an apex 641 and start points 643 and 645 of valves of the left atrium, in addition to the apex 621 of the left ventricle, the start points 623 and 625 of the mitral valves, the apex 631 of the right ventricle, and the start points 633 and 635 of the tricuspid valves. In addition, the ultrasound imaging apparatus 40 may determine an apex 651 and start points 653 and 655 of valves of the right atrium.

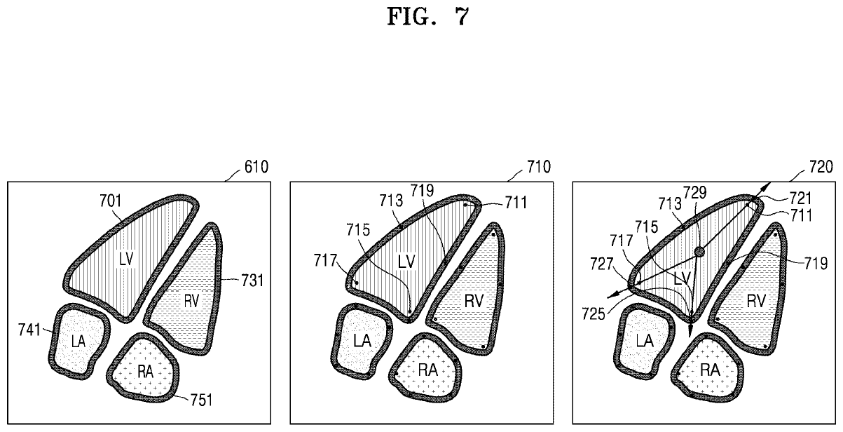

FIG. 7 illustrates a method, performed by the ultrasound imaging apparatus 40, of determining the apex and a valve of a chamber, according to an embodiment.

Referring to FIG. 7, the ultrasound imaging apparatus 40 may more accurately identify the apex and a valve of a chamber by using morphological features of the chamber.

The ultrasound imaging apparatus 40 may determine the apex and the valve of the chamber by using K-means clustering, which is an unsupervised learning algorithm. K-means clustering is a method of clustering data into a given number K of clusters. K-means clustering is a method of clustering data such that the sum of the squares of the distances between data points and the centroid of the cluster to which the data belongs is minimized.

Referring to the first image in FIG. 7, the ultrasound imaging apparatus 40 may obtain the chamber segmentation image 610 that distinguishably indicates the regions of the chambers.

The ultrasound imaging apparatus 40 may determine contours 701, 731, 741, and 751 of the respective chamber regions on the chamber segmentation image 610.

Referring to the second image (710) in FIG. 7, the ultrasound imaging apparatus 40 may cluster the points of the contour into k clusters according to a K-means clustering method, for each chamber. For example, k may be 5, and the value of k may be experimentally determined such that the centroids of the clusters are calculated to be close to the apex and the start points of valves.

For example, when k is 5, the ultrasound imaging apparatus 40 may randomly divide the points of the contour 701 of the left ventricle into 5 clusters. In addition, the ultrasound imaging apparatus 40 may calculate the centroid of each cluster. The points of the contour 701 may be iteratively reassigned to 5 clusters to reduce the sum of the squares of the distances from the points to their corresponding cluster centroids.

When the sum of the squares of the distances from the points of the contour 701 to their corresponding centroids is minimized, the ultrasound imaging apparatus 40 may obtain a centroid 711 near the apex, and centroids 715 and 717 near the start points of the valves, as illustrated in the second image (710) in FIG. 7. In addition, the ultrasound imaging apparatus 40 may obtain centroids 713 and 719 at the center of the inner wall of the left ventricle.

Because the apex and the start points of the valves have a higher curvature compared to other points of the contour, the centroids of the K-means clustering may be located near the apex and the start points of the valves in the direction toward the center of the chamber.

Referring to the third image (720) in FIG. 7, the ultrasound imaging apparatus 40 may determine, as candidate points of the left ventricle, points 713, 719, 721, 725, and 727, where line segments connecting the centroids 711, 713, 715, 717, and 719 of the clusters from a center 729 of the left ventricle region intersect the contour 701 of the left ventricle.

In addition, the ultrasound imaging apparatus 40 may determine the apex and the start point of the valve from among the candidate points 713, 719, 721, 725, and 727 based on the positions of the left atrium and the right atrium. For example, the ultrasound imaging apparatus 40 may determine, as the start point of the first mitral valve, the point 725 that is closest to the right atrium among the candidate points 713, 719, 721, 725, and 727 of the left ventricle. In addition, the ultrasound imaging apparatus 40 may determine, as the start point of the second mitral valve, the point 727 that is farther from the right atrium among the two points 725 and 727 close to the left atrium, from among the candidate points 713, 719, 721, 725, and 727 of the left ventricle. In addition, the ultrasound imaging apparatus 40 may determine, as the apex of the left ventricle, the point 721 that is farthest from the left atrium among the candidate points 713, 719, 721, 725, and 727 of the left ventricle.

Similar to the left ventricle, the ultrasound imaging apparatus 40 may determine the apex and valves of each of the right ventricle, the right atrium, and the left atrium through K-means clustering.

The positions of the apex and the valves may be detected more accurately by first detecting candidate points by using curvature, which is a morphological feature of the apex and valves, and then detecting the apex and valves from the candidate points using the relative positions of the chambers, rather than detecting the apex and valves by using only the relative positions of the chambers. For example, the ultrasound imaging apparatus 40 may accurately detect the positions of the apex and the valves even when the heart structure has an atypical shape or when there is significant image noise.

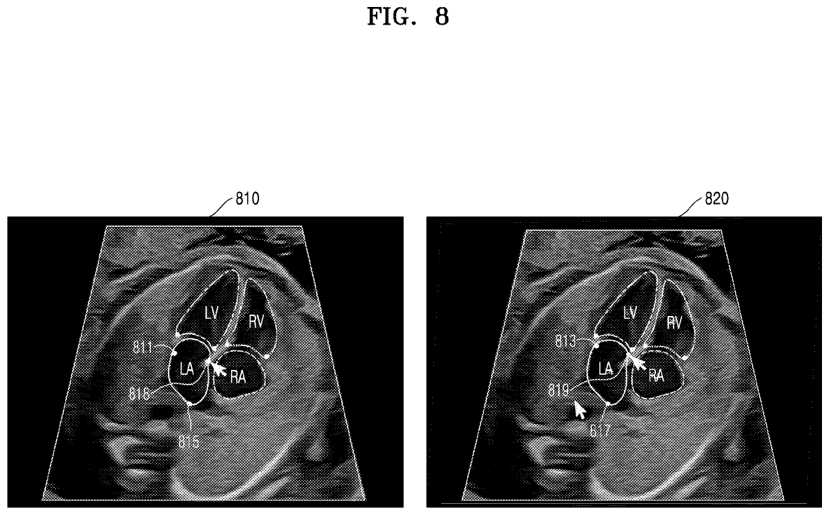

FIG. 8 illustrates a method, performed by the ultrasound imaging apparatus 40, of adjusting the positions of an apex and a valve based on a user input, according to an embodiment.

Referring to FIG. 8, the ultrasound imaging apparatus 40 may display the positions of a valve and an apex on an ultrasound image, and receive a user input for moving the displayed positions of the valve and apex.

For example, the ultrasound imaging apparatus 40 may provide an option for adjusting the position of a valve or an apex. Based on receiving a user input for selecting the option for adjusting the position of a valve or an apex, the ultrasound imaging apparatus 40 may provide a user interface for adjusting the position of a valve or an apex.

The ultrasound imaging apparatus 40 may receive a user input for selecting one of valves and apexes on the ultrasound image. For example, referring to the first diagram (810), the ultrasound imaging apparatus 40 may receive a user input of mouse-clicking or touching one of the valves and apexes. In addition, for example, the ultrasound imaging apparatus 40 may receive a user input for selecting one of the valves and apexes based on a movement of a wheel or a trackball.

The ultrasound imaging apparatus 40 may provide a user interface in which a valve or an apex moves along the contour of the chamber. For example, in response to receiving a user input, such as moving a mouse, wheel, or trackball, dragging the selected apex or valve, or selecting a directional key, the ultrasound imaging apparatus 40 may move the selected apex or valve along the contour of the chamber.

Referring to the second diagram (820) in FIG. 8, the ultrasound imaging apparatus 40 may receive a user input for moving the apex of the left atrium from a first position 815 to a second position 817. In addition, the ultrasound imaging apparatus 40 may receive a user input for moving a first mitral valve of the left atrium from a third position 811 to a fourth position 813. In addition, the ultrasound imaging apparatus 40 may receive a user input for moving a second mitral valve of the left atrium from a fifth position 818 to a sixth position 819.

The ultrasound imaging apparatus 40 may receive a user input for storing the moved position of the apex or valve. The ultrasound imaging apparatus 40 may change the position of the apex or valve in previous and subsequent frames based on the moved position of the apex or valve. For example, when the positions of the apex and valve of the left atrium are moved, the ultrasound imaging apparatus 40 may determine, as the position of the apex or valve of the left atrium, a point in other frames corresponding to the moved point. In addition, the ultrasound imaging apparatus 40 may recalculate the longitudinal strain of the chamber based on the changed positions of the apex and valve.

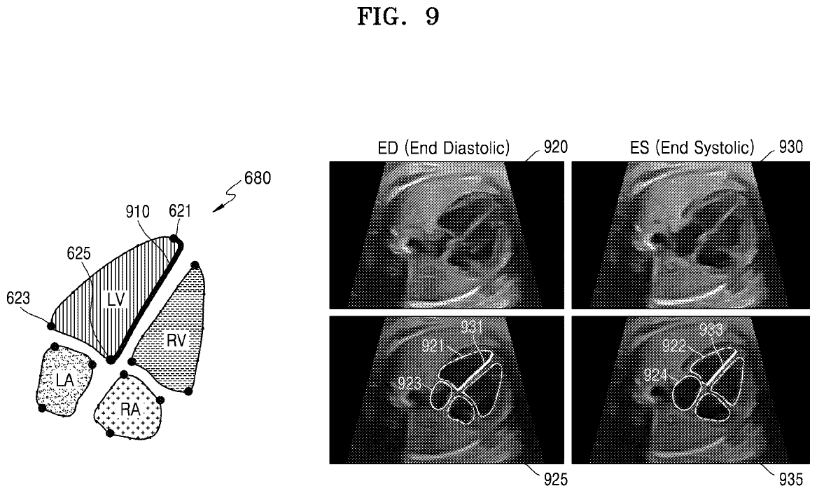

FIG. 9 illustrates a method, performed by the ultrasound imaging apparatus 40, of calculating a longitudinal strain of a chamber, according to an embodiment.

Referring to FIG. 9, the ultrasound imaging apparatus 40 may calculate a GLS, which is the longitudinal rate of change of the length between the apex and a valve from end-diastole to end-systole.

$$GLS = ( \frac{{L}_{ES} - {L}_{ED}}{{L}_{ED}} ) \times 100 \qquad Equation ( 1 )$$

In Equation (1) for longitudinal strain, LES refers to the length between the apex and the start point of the valve at end-systole, and LED may refer to the length between the apex and the start point of the valve at end-diastole.

The end-diastole is the time point when diastole ends, and may be the time point when the volume of the ventricle is largest. In addition, the end-systole is the time point when systole ends, and may be the time point when the volume of the ventricle is smallest. Referring to ultrasound images 920 and 925 at end-diastole, it may be confirmed that the volume of a left ventricle 921 is at its maximum, and the volume of a left atrium 923 is at its minimum. In addition, referring to ultrasound images 930 and 935 at end-systole, it may be confirmed that the volume of a left ventricle 922 is at its minimum, and the volume of a left atrium 924 is at its maximum.

The ultrasound imaging apparatus 40 may calculate the longitudinal strain of the left ventricle. For example, the ultrasound imaging apparatus 40 may calculate the longitudinal strain of the left ventricle by calculating a length 931 between the apex 621 of the left ventricle and the start point 625 of the first mitral valve at end-diastole, calculating a length 933 between the apex 621 of the left ventricle and the start point 625 of the first mitral valve at end-systole, and substituting the two lengths 931 and 933 into Equation (1). According to an embodiment, the ultrasound imaging apparatus 40 may also determine, as the longitudinal strain of the left ventricle, the strain of the length between the apex 621 and the start point 623 of the second mitral valve, instead of a length 910 between the apex 621 of the left ventricle and the start point 625 of the first mitral valve.

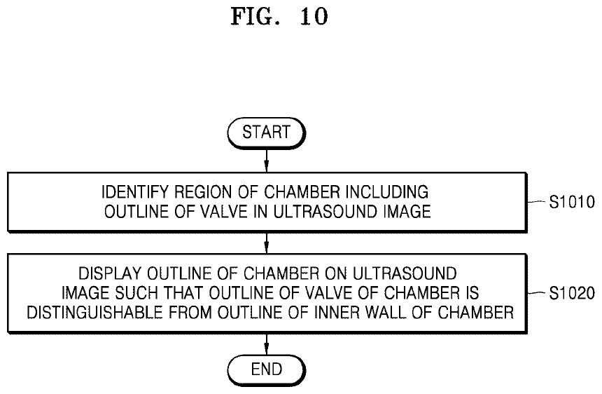

FIG. 10 is a flowchart of a method, performed by the ultrasound imaging apparatus 40, of identifying a contour of a valve, according to an embodiment.

In operation S1010, the ultrasound imaging apparatus 40 may identify a region of a chamber including the contour of a valve in an ultrasound image.

The ultrasound imaging apparatus 40 may determine the contour of the chamber such that it includes not only the contour of the inner wall of the chamber but also the contour of the valve of the chamber. For example, an AI model may be trained to output, when an ultrasound image is input, a region of a chamber, which includes the contour of the inner wall of the chamber and the contour of a valve of the chamber.

Chamber view images may be set as input data for training data, and images in which regions of the left ventricle, the right ventricle, the left atrium, and the right atrium within the chamber view images are indicated may be set as target data for the training data. In this case, the region of the chamber may be a region enclosed by the contour of the inner wall of the chamber and the contour of the valve of the chamber.

By training the AI model based on a sufficient amount of training data, the AI model may output the region of a chamber including the contour of the inner wall of the chamber and the contour of a valve of the chamber.

In operation S1020, the ultrasound imaging apparatus 40 may display the contour of the chamber on the ultrasound image such that the contour of the valve of the chamber is distinguishable from the contour of the inner wall of the chamber.

By displaying the contour of the valve of the chamber distinguishably from the contour of the inner wall of the chamber, a user may intuitively check a movement of the valve.

In addition, by displaying the contour of the valve of the chamber distinguishably from the contour of the inner wall of the chamber, the user may check whether the valve is open or closed. For example, when the contour of the valve is indented toward the center of the chamber, the user may determine that the valve is open.

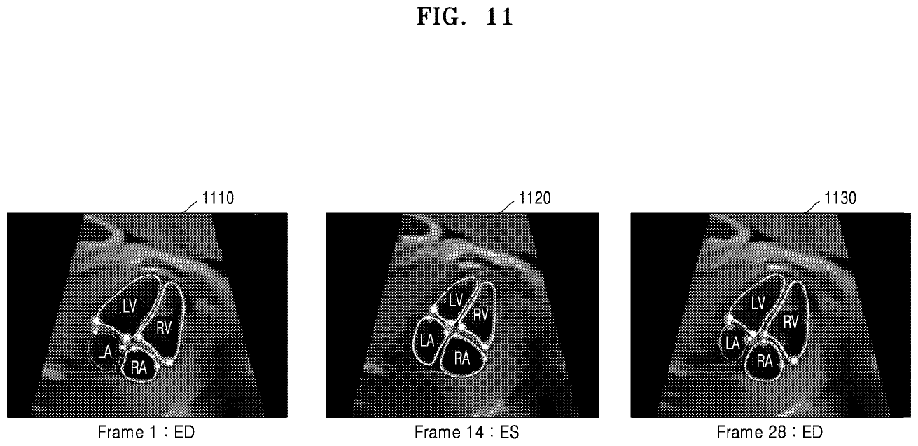

FIG. 11 illustrates a method, performed by the ultrasound imaging apparatus 40, of determining a cycle of a heart by considering the contour of a valve, according to an embodiment.

Referring to FIG. 11, the ultrasound imaging apparatus 40 may determine the sizes of chambers by considering the contours of valves, and determine end-systole and end-diastole of the heart based on the sizes of the chambers.

The ultrasound imaging apparatus 40 may determine the contour of the chamber such that it includes not only the contour of the inner wall of the chamber but also the contour of the valve of the chamber.

The ultrasound imaging apparatus 40 may determine, as the end-diastole of the heart, the time point when the size of the left ventricle is largest. In addition, the ultrasound imaging apparatus 40 may determine, as the end-systole of the heart, the time point when the size of the left atrium is largest.

The ultrasound imaging apparatus 40 may obtain ultrasound images of the object's heart, and calculate the size of the left ventricle and the size of the left atrium for each of the obtained ultrasound images. The ultrasound imaging apparatus 40 may determine a waveform representing the size of the left ventricle over the obtained ultrasound images, and determine, as the end-diastole, the time points corresponding to peaks in the waveform of the size of the left ventricle. Referring to FIG. 11, it may be determined that the waveform of the size of the left ventricle indicates peaks at frame 1 1110 and frame 28 1130 among the obtained ultrasound images.

In addition, the ultrasound imaging apparatus 40 may determine a waveform representing the size of the left atrium over the obtained ultrasound images, and determine, as the end-systole, the time points corresponding to peaks in the waveform of the size of the left atrium. The ultrasound imaging apparatus 40 may determine one end-systole between two adjacent end-diastoles. Referring to FIG. 11, it may be determined that the waveform of the size of the left atrium indicates a peak at frame 14 1120 among the obtained ultrasound images.

The ultrasound imaging apparatus 40 may calculate the size of the chamber more accurately by calculating the size of the chamber based on the contour of the valve that regulates blood flow entering or exiting the chamber. By accurately calculating the size of the chamber, the ultrasound imaging apparatus 40 may accurately detect the end-diastole and the end-systole.

As illustrated in FIG. 11, the ultrasound imaging apparatus 40 may display identification information of a left ventricle LV, a left atrium LA, a right ventricle RV, and a right atrium RA on the ultrasound image.

In addition, the ultrasound imaging apparatus 40 may display the valve of the chamber distinguishably from the inner wall of the chamber. For example, as illustrated in FIG. 11, the ultrasound imaging apparatus 40 may display the inner wall of the chamber as a solid line and the contour of the valve of the chamber as a dotted line. Accordingly, a user may accurately check a movement of the valve, and may accurately distinguish between systole and diastole by checking not only the size of the chamber but also the shape of the valve.

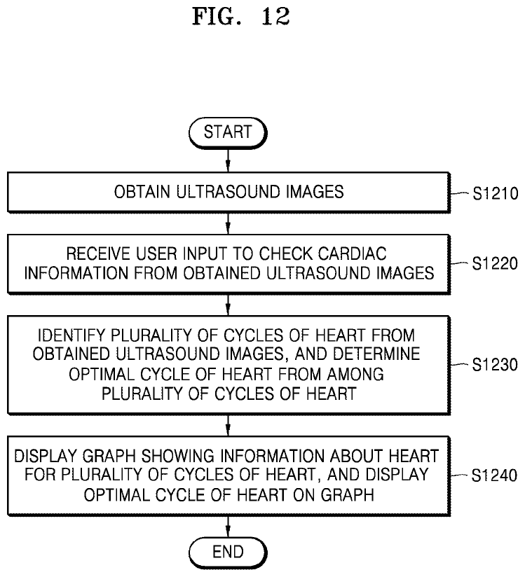

FIG. 12 is a flowchart of a method, performed by the ultrasound imaging apparatus 40, of providing information about a cycle of a heart, according to an embodiment.

In operation S1210, the ultrasound imaging apparatus 40 may obtain ultrasound images.

When the heart rate of a fetus is about 2 to 3 beats per second, and the fetal heart is imaged for 4 seconds, the ultrasound imaging apparatus 40 may obtain ultrasound images showing about 8 to 12 cycles of the heart. In addition, operation S1210 may be described with reference to operation S410 of FIG. 4.

In operation S1220, the ultrasound imaging apparatus 40 may receive a user input to check cardiac information from the obtained ultrasound images.

The ultrasound imaging apparatus 40 may receive a user input selecting a hardware button or a software button for checking cardiac information from the obtained ultrasound images.

In operation S1230, the ultrasound imaging apparatus 40 may identify a plurality of cycles of the heart from the obtained ultrasound images, and determine an optimal cycle of the heart from among the plurality of cycles of the heart.

Based on receiving the user input to check cardiac information from the obtained ultrasound images, the ultrasound imaging apparatus 40 may identify the ultrasound images indicating an end-diastole or an end-systole among the obtained ultrasound images. The ultrasound imaging apparatus 40 may identify the time points of the plurality of cycles of the heart based on the ultrasound images indicating the end-diastole or the end-systole.

In addition, the ultrasound imaging apparatus 40 may determine the optimal cycle of the heart from among the plurality of cycles of the heart.

According to an embodiment, the ultrasound imaging apparatus 40 may determine, as the optimal cycle of the heart, the cycle having the largest amount of change in the longitudinal strain of the left ventricle among the plurality of cycles of the heart. For example, the ultrasound imaging apparatus 40 may determine, as the optimal cycle, the cycle of the heart having the largest difference between the longitudinal strain at end-diastole and the longitudinal strain at end-systole.

According to an embodiment, the ultrasound imaging apparatus 40 may also determine, as the optimal cycle of the heart, the cycle having the largest rate of change in the area of the left ventricle among the plurality of cycles of the heart. For example, the ultrasound imaging apparatus 40 may also determine, as the optimal cycle, the cycle of the heart having the largest difference between the area of the left ventricle at an end-diastole and the area of the left ventricle at an end-systole.

In operation S1240, the ultrasound imaging apparatus 40 may display a graph showing information about the heart for the plurality of cycles of the heart, and display the optimal cycle of the heart on the graph.

The graph showing information about the heart may include, but is not limited to, at least one of a graph of the areas of the chambers, a graph of the longitudinal strains of the chambers, a graph of FACs of the chambers, a graph of EFs of the chambers, and a graph of GLSRs of the chambers.

The ultrasound imaging apparatus 40 may display the optimal cycle of the heart distinguishably from the other cycles of the heart on the graph showing the information about the heart.

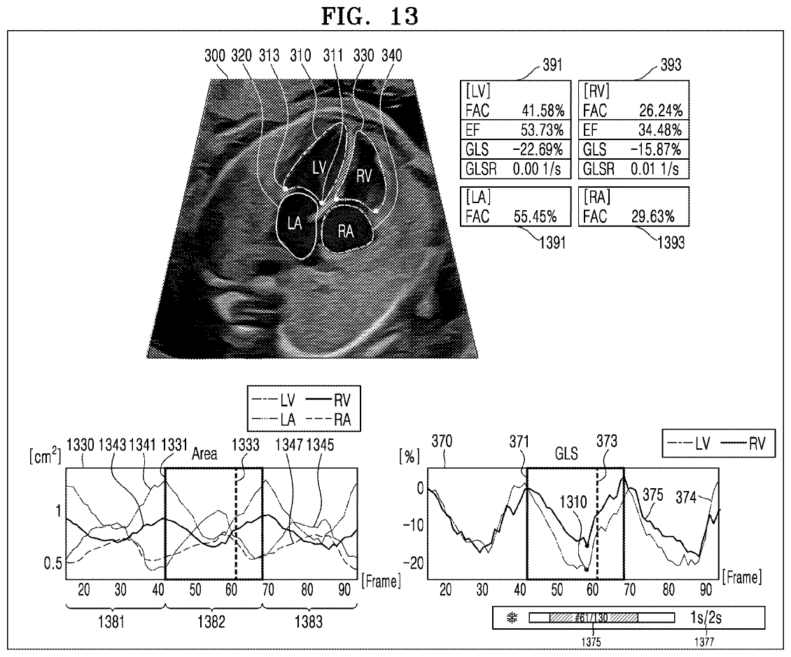

FIG. 13 is a diagram illustrating a method, performed by the ultrasound imaging apparatus 40, of displaying a plurality of cycles of a heart, according to an embodiment.

Referring to FIG. 13, the ultrasound imaging apparatus 40 may display a graph for the plurality of cycles of the heart, and recommend an optimal cycle of the heart among the plurality of cycles of the heart.

The ultrasound imaging apparatus 40 may obtain ultrasound images of an object's heart. The ultrasound imaging apparatus 40 may obtain about 200 frames of B-mode ultrasound images for 2 seconds based on set imaging parameters. For a fetus having a heart rate of about 3 beats per second, about 3 cycles of the heart may occur within 1 second.

The ultrasound imaging apparatus 40 may generate a graph of chamber area from the obtained ultrasound images. Based on the generated graph of chamber area, the ultrasound imaging apparatus 40 may determine the cycle of the object's heart. For example, the ultrasound imaging apparatus 40 may determine, as the end-diastole, the peak points in a graph 1341 of the area of the left ventricle. For example, in the graph 1341 of the area of the left ventricle in FIG. 13, the 1st, 42nd, 68th, and 94th frames may be determined as the frames corresponding to the end-diastole. In addition, the ultrasound imaging apparatus 40 may determine, as the end-systole, the peak points in a graph 1345 of the area of the left atrium. For example, in the graph 1345 of the area of the left atrium in FIG. 13, the 29th, 55th, and 84th frames may be determined as the frames corresponding to the end-systole.

The ultrasound imaging apparatus 40 may determine, as one cycle of the heart, the period from one end-diastole to the next end-diastole. The ultrasound imaging apparatus 40 may also determine, as one cycle of the heart, the period from one end-systole to the next end-systole.

The ultrasound imaging apparatus 40 may determine a plurality of cycles of the heart appearing in the obtained ultrasound images. For example, the ultrasound imaging apparatus 40 may determine the frame interval between frame 1 and frame 41 as a first cycle 1381, determine the frame interval between frame 42 and frame 67 as a second cycle 1382, and determine the frame interval between frame 68 and frame 94 as a third cycle 1383.

The ultrasound imaging apparatus 40 may receive a user input for selecting an option to check cardiac information after imaging the object's heart. Based on receiving the user input for selecting the option to check cardiac information, the ultrasound imaging apparatus 40 may display a graph for the plurality of cycles 1381, 1382, and 1383 of the heart, and recommend the optimal cycle 1382 of the heart among the plurality of cycles of the heart. For example, the ultrasound imaging apparatus 40 may determine, as the optimal cycle 1382 of the heart, the cycle having the largest amount of change in the longitudinal strain of the left ventricle among the plurality of cycles 1381, 1382, and 1383 of the heart. Referring to FIG. 13, because the longitudinal strain of the left ventricle shows a minimum value 1310 at frame 58, and the difference between the maximum value and the minimum value of the longitudinal strain of the left ventricle is largest in the second cycle 1382 to which frame 58 belongs, the ultrasound imaging apparatus 40 may determine the second cycle 1382 as the optimal cycle of the heart.

The ultrasound imaging apparatus 40 may display the graph for the plurality of cycles 1381, 1382, and 1383 of the heart, and display the optimal cycle 1382 of the heart distinguishably from the other cycles. For example, the ultrasound imaging apparatus 40 may display focus boxes 1331 and 371 on the optimal cycle 1382 of the heart. In addition, for example, the ultrasound imaging apparatus 40 may display the optimal cycle 1382 of the heart to be brighter than the other cycles.

The ultrasound imaging apparatus 40 may display cardiac performance information for the optimal cycle 1382 of the heart. The cardiac performance information may include, but is not limited to, FACs, EFs, GLSs, and GLSRs of the chambers.

For example, the ultrasound imaging apparatus 40 may display cardiac performance information 391 for the left ventricle, cardiac performance information 393 for the right ventricle, cardiac performance information 1391 for the left atrium, and cardiac performance information 1393 for the right atrium, corresponding to the optimal cycle 1382 of the heart.

According to an embodiment, the ultrasound imaging apparatus 40 may display a chamber area graph 1330 including a left ventricle area graph 1341, a right ventricle area graph 1343, a left atrium area graph 1345, and a right atrium area graph 1347.

According to an embodiment, the ultrasound imaging apparatus 40 may display the longitudinal strain graph 370 including the left ventricle longitudinal strain graph 374 and the right ventricle longitudinal strain graph 375.

According to an embodiment, the ultrasound imaging apparatus 40 may display a playback interval 1377 of the ultrasound images used for analysis, within the entire playback duration of the obtained ultrasound images. In addition, the ultrasound imaging apparatus 40 may display a frame number 1375 of the currently displayed ultrasound image 300, relative to the total number of frames of the ultrasound images used for analysis.

According to an embodiment, the ultrasound imaging apparatus 40 may display IDs 1333 and 373 indicating the position of the currently displayed ultrasound image 300, on the chamber area graph 1330 and the longitudinal strain graph 370, respectively. Accordingly, a user may check where the currently displayed ultrasound image is located within the cycle of the heart. In addition, the user may check the areas of the chambers or the longitudinal strains of the chambers in the current ultrasound image.

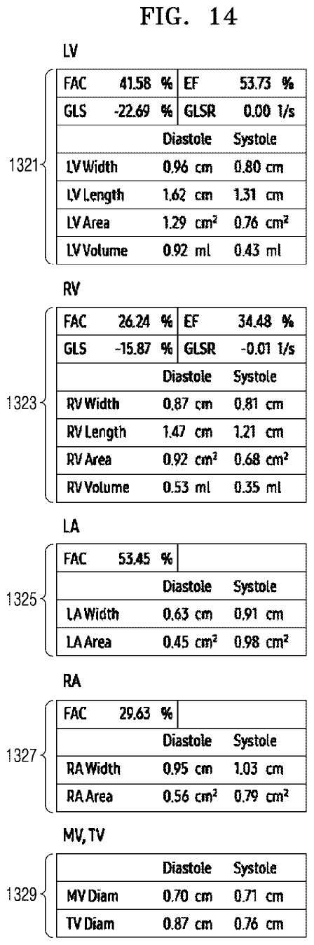

FIG. 14 illustrates a method, performed by the ultrasound imaging apparatus 40, of displaying information about chambers, according to an embodiment.

Referring to FIG. 14, the ultrasound imaging apparatus 40 may display information about the chambers at the end-diastole and end-systole of the optimal cycle 1382 (see FIG. 13) of the heart. The information about the chamber may include, but is not limited to, at least one of width, length, area, or volume. For example, the ultrasound imaging apparatus 40 may display information 1321 about the left ventricle, information 1323 about the right ventricle, information 1325 about the left atrium, and information 1327 about the right atrium at the end-diastole and end-systole of the optimal cycle 1382 (see FIG. 13) of the heart.

In addition, the ultrasound imaging apparatus 40 may display information about valves at the end-diastole and end-systole of the optimal cycle 1382 (see FIG. 13) of the heart. The information about the valve may include, but is not limited to, the diameter of the valve. For example, the ultrasound imaging apparatus 40 may display the diameter of a mitral valve MV and the diameter of a tricuspid valve TV (1329) at the end-diastole and end-systole of the optimal cycle 1382 (see FIG. 13) of the heart.

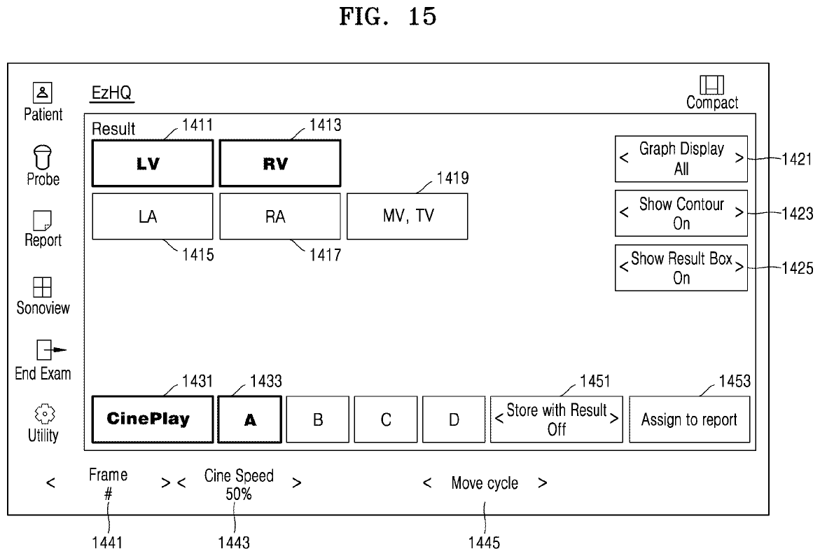

FIG. 15 illustrates a user interface for cardiac information, according to an embodiment.

Referring to FIG. 15, the ultrasound imaging apparatus 40 may display a user interface for setting a method of displaying cardiac information.

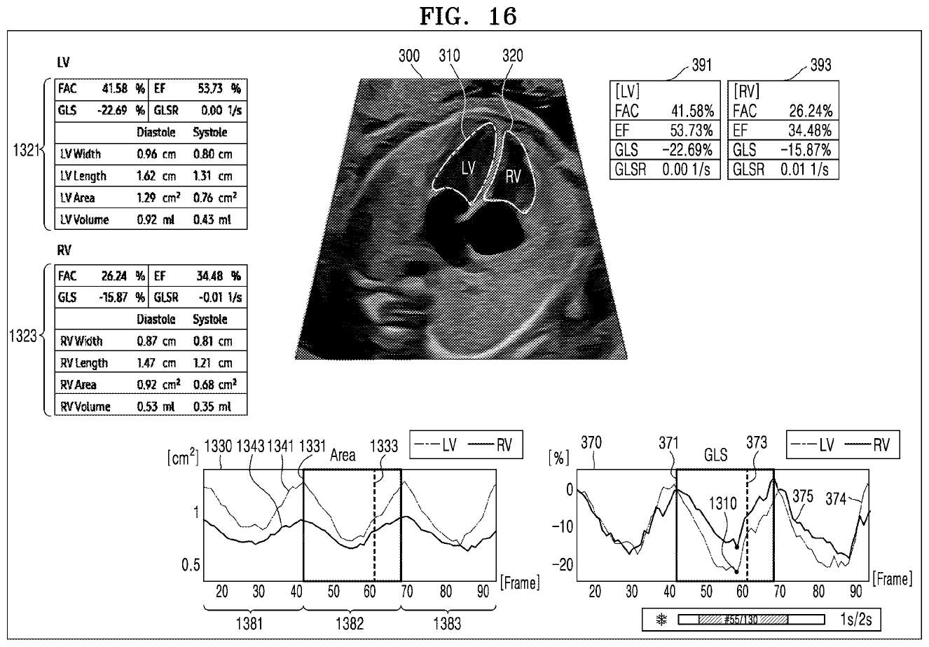

According to an embodiment, the ultrasound imaging apparatus 40 may display an ultrasound image and cardiac information as illustrated in FIG. 13 or 16 on the main display 121 (see FIGS. 2A and 2B), and display the user interface for setting the method of displaying cardiac information of FIG. 15 on the sub-display 122 (see FIGS. 2A and 2B).

According to an embodiment, in a case in which the ultrasound imaging apparatus 40 is a portable ultrasound imaging apparatus 40 (see FIG. 2C or 2D), the ultrasound imaging apparatus 40 may display the ultrasound image and the cardiac information together with the user interface on a single display.

Referring to FIG. 15, the ultrasound imaging apparatus 40 may display user interfaces 1411, 1413, 1415, 1417, and 1419 for selecting a chamber or valve for which to obtain cardiac information from among a plurality of chambers and valves. Based on receiving a user input for selecting the left ventricle button 1411 and the right ventricle button 1413 from among the plurality of chamber buttons 1411, 1413, 1415, and 1417 and the valve button 1419, the ultrasound imaging apparatus 40 may display only information about the left ventricle and the right ventricle.

The ultrasound imaging apparatus 40 may display a user interface 1421 for selecting the type of graph to be provided. For example, the ultrasound imaging apparatus 40 may receive, via the user interface 1421, a user input for selecting at least one of a chamber area graph or a longitudinal strain graph.

The ultrasound imaging apparatus 40 may display a user interface 1423 for selecting whether to display the contours of the chambers.

The ultrasound imaging apparatus 40 may provide a user interface 1425 for selecting whether to display a performance indicator for the heart in a separate area near the ultrasound image.

The ultrasound imaging apparatus 40 may display a user interface 1431 for continuously playing back the ultrasound images. In addition, in a case in which the object involves a plurality of fetuses, the ultrasound imaging apparatus 40 may display a user interface 1433 for selecting the fetus to be measured from among the plurality of fetuses. In addition, the ultrasound imaging apparatus 40 may display a user interface 1451 for selecting whether to store a measurement result together with the ultrasound image. In addition, the ultrasound imaging apparatus 40 may display a user interface 1453 for storing measurements, performance indicators for the heart, and ultrasound images in a patient report format.

The ultrasound imaging apparatus 40 may display a frame number 1441 of the ultrasound image currently being displayed. The ultrasound imaging apparatus 40 may display a user interface 1443 for selecting a playback speed when continuously playing back the ultrasound images. The ultrasound imaging apparatus 40 may display a user interface 1445 for changing the currently focused cycle to the previous or next cycle.

FIG. 16 illustrates a method, performed by the ultrasound imaging apparatus 40, of providing information about a heart according to user settings, according to an embodiment.

Referring to FIG. 16, the ultrasound imaging apparatus 40 may display information about a heart according to user settings that are input via the user interfaces illustrated in FIG. 15.

Based on receiving a user input for selecting the left ventricle button 1411 (see FIG. 15) and the right ventricle button 1413 (see FIG. 15) from among the plurality of chamber buttons, the ultrasound imaging apparatus 40 may calculate performance indicators and measurements for the left ventricle, and performance indicators and measurements for the right ventricle. In addition, the ultrasound imaging apparatus 40 may display performance indicators 391 and measurements 1321 for the left ventricle, and performance indicators 393 and measurements 1323 for the right ventricle.

In addition, based on setting via the user interface 1423 (see FIG. 15) to display the contours of the chambers, the ultrasound imaging apparatus 40 may display the contour of the left ventricle 310 and the contour of the right ventricle 320 on the ultrasound image 300.

In addition, based on receiving, via the user interface 1421 (see FIG. 15), a user input for selecting both the chamber area graph and the longitudinal strain graph as the types of graphs to be provided, the ultrasound imaging apparatus 40 may display the chamber area graph 1330 including the left ventricle area graph 1341 and the right ventricle area graph 1343, and the longitudinal strain graph 370 including the left ventricle longitudinal strain graph 374 and the right ventricle longitudinal strain graph 375.

By displaying contours and graphs in different colors for different chambers, a user may easily distinguish between pieces of information about a plurality of chambers. For example, the contour of the left ventricle 310, the left ventricle area graph 1341, and the left ventricle longitudinal strain graph 374 may be represented in red, while the contour of the right ventricle 320, the right ventricle area graph 1343, and the right ventricle longitudinal strain graph 375 may be represented in blue.

Based on setting via the user interface 1425 (see FIG. 15) to display the performance indicators for the heart in a separate area near the ultrasound image, the ultrasound imaging apparatus 40 may display the performance indicators 391 for the left ventricle and the performance indicators 393 for the right ventricle, corresponding to the currently focused cycle 1382, near the ultrasound image 300.

Based on receiving, via the user interface 1431 (see FIG. 15), a user input for playing back the ultrasound images, the ultrasound imaging apparatus 40 may play back the ultrasound images corresponding to the focused cycle 1382. In addition, as the ultrasound images are played back, the identifiers 1333 and 373, which indicate the position of the currently displayed ultrasound image within the graphs 1330 and 370, may be moved within the focus boxes 1331 and 371.

The ultrasound imaging apparatus 40 may display the cycle to which the currently displayed ultrasound image 300 belongs distinguishably from the other cycles in the chamber area graph 1330 and the longitudinal strain graph 370. For example, the ultrasound imaging apparatus 40 may display the focus boxes 1331 and 371 on the cycle 1382 to which the currently displayed ultrasound image 300 belongs. In addition, for example, the ultrasound imaging apparatus 40 may display the cycle 1382, to which the currently displayed ultrasound image 300 belongs, to be brighter than the other cycles.

A machine-readable storage medium may be provided in the form of a non-transitory storage medium. Here, the term ‘non-transitory storage medium’ refers to a tangible device and does not include a signal (e.g., an electromagnetic wave), and the term ‘non-transitory storage medium’ does not distinguish between a case where data is stored in a storage medium semi-permanently and a case where data is stored temporarily. For example, the ‘non-transitory storage medium’ may include a buffer in which data is temporarily stored.

According to an embodiment, methods according to various embodiments disclosed herein may be included in a computer program product and then provided. The computer program product may be traded as a commodity between sellers and buyers. The computer program product may be distributed in the form of a machine-readable storage medium (e.g., a compact disc read-only memory (CD-ROM)), or may be distributed online (e.g., downloaded or uploaded) through an application store or directly between two user devices (e.g., smart phones). In a case of online distribution, at least a portion of the computer program product (e.g., a downloadable app) may be temporarily stored in a machine-readable storage medium such as a manufacturer's server, an application store's server, or memory of a relay server.

What is claimed is:

1. An ultrasound imaging apparatus comprising:
an ultrasound transceiver module;
a memory storing instructions; and
at least one processor comprising processing circuitry,
wherein the instructions, when executed by the at least one processor individually or collectively, cause the ultrasound imaging apparatus to emit, via the ultrasound transceiver module, an ultrasound signal toward a heart of an object, obtain ultrasound images of a cycle of the heart based on an ultrasound echo signal received from the object via the ultrasound transceiver module, identify regions of a plurality of chambers in the obtained ultrasound images, determine a position of an apex of a first chamber among the plurality of chambers and a position of a valve of the first chamber, based on a relative position between a second chamber and the apex and the valve of the first chamber, calculate information about the heart based on the position of the apex and the position of the valve, and display the calculated information.

2. The ultrasound imaging apparatus of claim 1, wherein the first chamber comprises a left ventricle, the second chamber comprises a left atrium, and
the instructions, when executed by the at least one processor individually or collectively, cause the ultrasound imaging apparatus to determine, as the position of the apex of the left ventricle, a point in a region of the left ventricle that is farthest from the left atrium.

3. The ultrasound imaging apparatus of claim 1, wherein the first chamber comprises a left ventricle, the second chamber comprises a right atrium, and
the instructions, when executed by the at least one processor individually or collectively, cause the ultrasound imaging apparatus to determine, as the position of the valve, a point in a region of the left ventricle that is closest to the right atrium.

4. The ultrasound imaging apparatus of claim 1, wherein the instructions, when executed by the at least one processor individually or collectively, cause the ultrasound imaging apparatus to display the position of the apex of the first chamber and the position of the valve, on an ultrasound image, from among the obtained ultrasound images, in which the position of the apex and the position of the valve have been identified.

5. The ultrasound imaging apparatus of claim 4, wherein the instructions, when executed by the at least one processor individually or collectively, cause the ultrasound imaging apparatus to receive a user input for selecting one of the displayed apex and valve and adjusting the position of the selected apex or valve, and recalculate the information about the heart based on the adjusted position of the apex or the valve.

6. The ultrasound imaging apparatus of claim 1, wherein the cycle of the heart comprises a plurality of cycles of the heart, and
the instructions, when executed by the at least one processor individually or collectively, cause the ultrasound imaging apparatus to display a graph showing the information about the heart during the plurality of cycles.

7. The ultrasound imaging apparatus of claim 6, wherein the instructions, when executed by the at least one processor individually or collectively, cause the ultrasound imaging apparatus to determine an optimal cycle from among the plurality of cycles of the heart, and display the determined optimal cycle on the graph showing the information about the heart during the plurality of cycles.

8. The ultrasound imaging apparatus of claim 1, wherein the ultrasound images comprise two or more ultrasound images in which orientations of the heart differ by at least a reference angle.

9. The ultrasound imaging apparatus of claim 1, wherein the instructions, when executed by the at least one processor individually or collectively, cause the ultrasound imaging apparatus to identify, in the obtained ultrasound images, regions of the first chamber, which include a contour of the valve of the first chamber.

10. The ultrasound imaging apparatus of claim 9, wherein the instructions, when executed by the at least one processor individually or collectively, cause the ultrasound imaging apparatus to display one ultrasound image from among the ultrasound images, and display a contour of the first chamber on the displayed ultrasound image, such that the contour of the valve of the first chamber is distinguished from a contour of an inner wall of the first chamber.

11. A method of providing information about a heart from a cardiac ultrasound image, the method comprising:
emitting an ultrasound signal toward a heart of an object and obtaining ultrasound images of a cycle of the heart based on an ultrasound echo signal received from the object;
identifying regions of a plurality of chambers in the obtained ultrasound images;
determining a position of an apex of a first chamber among the plurality of chambers and a position of a valve of the first chamber, based on a relative position between a second chamber and the apex and the valve of the first chamber;
calculating information about the heart based on the position of the apex and the position of the valve; and
displaying the calculated information.

12. The method of claim 11, wherein the first chamber comprises a left ventricle, the second chamber comprises a left atrium, and
the determining of the position of the apex of the first chamber among the plurality of chambers and the position of the valve of the first chamber, based on the relative position between the second chamber and the apex and the valve of the first chamber, comprises determining, as the position of the apex of the left ventricle, a point in a region of the left ventricle that is farthest from the left atrium.

13. The method of claim 11, wherein the first chamber comprises a left ventricle, the second chamber comprises a right atrium, and
the determining of the position of the apex of the first chamber among the plurality of chambers and the position of the valve of the first chamber, based on the relative position between the second chamber and the apex and the valve of the first chamber, comprises determining, as the position of the valve, a point in a region of the left ventricle that is closest to the right atrium.

14. The method of claim 11, further comprising displaying the position of the apex of the first chamber and the position of the valve, on an ultrasound image, from among the obtained ultrasound images, in which the position of the apex and the position of the valve have been identified.

15. The method of claim 14, further comprising:
receiving a user input for selecting one of the displayed apex and valve and adjusting the position of the selected apex or valve; and
recalculating the information about the heart based on the adjusted position of the apex or the valve.

16. The method of claim 11, wherein the cycle of the heart comprises a plurality of cycles of the heart, and
the displaying of the calculated information comprises displaying a graph showing the information about the heart during the plurality of cycles.

17. The method of claim 16, wherein the displaying of the calculated information further comprises:
determining an optimal cycle from among the plurality of cycles of the heart; and
displaying the determined optimal cycle on the graph showing the information about the heart during the plurality of cycles.

18. The method of claim 11, wherein the ultrasound images comprise two or more ultrasound images in which orientations of the heart differ by at least a reference angle.

19. The method of claim 11, wherein the identifying of the regions of the plurality of chambers in the obtained ultrasound images comprises identifying, in the obtained ultrasound images, regions of the first chamber, which include a contour of the valve of the first chamber.

20. The method of claim 19, further comprising:
displaying one ultrasound image from among the ultrasound images; and
displaying the contour of the first chamber on the displayed ultrasound image, such that the contour of the valve of the first chamber is distinguished from a contour of an inner wall of the first chamber.
